(19) United States
(12) Patent Application Publication
(10) Pub. No.: US 2024/0199664 A1
(43) Pub. Date: Jun. 20, 2024

(54) CONDUCTIVE STIMULI-RESPONSIVE COORDINATION NETWORK LINKED WITH BISMUTH

(71) Applicants: Trustees of Dartmouth College, Hanover, NH (US); The Regents of the University of California, Oalkland, CA (US)

(72) Inventors: Katherine A. Mirica, Hanover, NH (US); Aylin Aykanat, Lyme, NH (US); Evan Cline, Lebanon, NH (US); Zheng Meng, West Lebanon, NH (US); Christopher G. Jones, Los Angeles (CA); Hosea Nelson, Venice, CA (US)

(73) Assignees: Trustees of Dartmouth College, Hanover, NH (US); The Regents of The University Of California, Oakland, CA (US)

(21) Appl. No.: 18/286,796

(22) PCT Filed: Apr. 12, 2022
(86) PCT No.: PCT/US2022/024462

§ 371 Date: Oct. 13, 2023

Related U.S. Application Data
(60) Provisional application No. 63/174,854, filed on Apr. 14, 2021.

(51) Int. Cl. C07F 9/94; A61B 6/10; G01N 31/22

(52) U.S. Cl. CPC C07F 9/94; A61B 6/107; G01N 31/22

(57) ABSTRACT

Embodiments of the present disclosure pertain to compositions that include a metal-organic framework. The metal-organic framework includes a plurality of metals and a plurality of ligands coordinated with the plurality of metals, where the plurality of metals include bismuth, and the plurality of ligands include a plurality of hydroxy moieties that are not part of carboxyl groups. At least some of the hydroxy moieties are coordinated with bismuth to form Bi—O bonds. The metal-organic framework is in the form of a conductive and interconnected network. Additional embodiments of the present disclosure pertain to methods of utilizing the compositions to detect analytes in a sample and shield various objects from radiation.

### CROSS-REFERENCE TO RELATED APPLICATIONS

The present application claims the benefit of U.S. Provisional Patent Application No. 63/174,854, filed on Apr. 14, 2021. The entirety of the aforementioned application is incorporated herein by reference.

### STATEMENT REGARDING FEDERALLY SPONSORED RESEARCH

This invention was made with government support under W911NF-17-1-0398 awarded by the Department of Defense, 1945218 awarded by the National Science Foundation, and R35GM138318 awarded by the National Institutes of Health. The government has certain rights in the invention.

### BACKGROUND

Crystalline conductive coordination polymers, such as metal-organic frameworks and coordination networks, offer promising alternatives as new emerging classes of materials with broad applicability. However, most conductive coordination polymers that have thus far been employed have significant shortcomings. Various embodiments of the present disclosure seek to address these shortcomings.

### SUMMARY

In some embodiments, the present disclosure pertains to compositions that include a metal-organic framework. In some embodiments, the metal-organic framework includes a plurality of metals and a plurality of ligands coordinated with the plurality of metals. In some embodiments, the plurality of metals include bismuth and the plurality of ligands include a plurality of hydroxy moieties that are not part of carboxyl groups. In some embodiments, at least some of the hydroxy moieties are coordinated with bismuth to form Bi—O bonds. In some embodiments, the metal-organic framework is in the form of a conductive and interconnected network.

In some embodiments, the metal-organic framework includes the following formula: M_{y}(HXTP)_{z}, where M includes bismuth, X is O or NH, y is 1 or 3, z is 1 or 2, and HXTP represents a triphenylene-based ligand that includes, without limitation, 2,3,5,6,10,11-hexahydroxytriphenylene (HHTP), 2,3,5,6,10,11 hexaiminotriphenylene (HITP), 2,3,5,6,10,11-hexathiotriphenylene (HTTP), and combinations thereof. In some embodiments, the metal-organic framework includes Bi(HHTP)-α with a 2,3-c nodal net stoichiometry. In some embodiments, the metal-organic framework includes Bi(HHTP)-β with a 3,4,4,5-c nodal net stoichiometry.

In some embodiments, the metal-organic framework is a component of a sensor or a dosimeter. In some embodiments, the metal-organic framework is associated with a textile. In some embodiments, the metal-organic framework is a component of a personal protective equipment.

Further embodiments of the present disclosure pertain to methods of detecting an analyte in a sample by associating the sample with a composition of the present disclosure; and detecting the presence or absence of the analyte from the sample. The detecting can occur by detecting a change in a property of the sensor and correlating the change in the property to the presence or absence of the analyte. In some embodiments, the exposing results in the reversible association of analytes in the sample with the composition. In some embodiments, the association also results in the capture of the analyte by the sensor. Additional embodiments of the present disclosure also include a step of releasing the analyte from the sensor.

Additional embodiments of the present disclosure pertain to methods of shielding an object from radiation by associating the object with a composition of the present disclosure. In some embodiments, the object is a human being, the composition is in the form of a personal protective equipment, and the associating occurs by wearing the personal protective equipment.

### BRIEF DESCRIPTION OF THE DRAWINGS

FIG. 1A illustrates a metal-organic framework in accordance with various embodiments of the present disclosure.

FIG. 1B depicts a method of detecting analytes in accordance with various embodiments of the present disclosure.

FIG. 1C depicts a method of shielding an object from radiation in accordance with various embodiments of the present disclosure.

FIGS. 2A-2D illustrate the synthesis of Bi(HHTP)-α. FIG. 2A illustrates the general reaction scheme for the synthesis. FIG. 2B provides the chemical structure of synthesized Bi(HHTP)-α, which exhibits oxidation states of the HHTP ligand. FIGS. 2C and 2D provide the structure of Bi(HHTP)-α showing a polyhedral (FIG. 2D) around two nonequivalent coordination polyhedral of Bi₁ as a distorted quadrilateral (CN=4) (top panel of FIG. 2D) and Bi₂ as a distorted tetragonal pyramid (CN=5, bottom panel of FIG. 2D).

FIGS. 3A-3B provide experimental and simulated pXRD patterns of Bi(HHTP)-α (FIG. 3A) and corresponding (002), (202), (200), (102), and (321) crystalline planes and interatomic distances (FIG. 3B).

FIGS. 4A-4C depict structures of Bi(HHTP)-α, including a Scanning Electron Micrograph (SEM) structure (FIG. 4A), a Transmission Electron Micrograph (TEM) structure (FIG. 4B), and a Selected Area Electron of Bi(HHTP)-α (FIG. 4C).

FIGS. 5A-5D depict the structures of Bi(HHTP)-α (FIG. 5A) and Bi(HHTP)-β (FIG. 5B), depicting two coordination polyhedral of Bi₁ and Bi₂. As illustrated in FIG. 5C, Bi(HHTP)-α displays a 2-nodal net of 2,3-C4 topological type obtained within standard representation of second model (Point symbol for net: [4.8.10] [8]). As depicted in FIG. 5D, Bi(HHTP)-β resulted in the 3,4,4,5-c nodal net of the new topological type with point symbol for net: {42.62.82} {42.6}{43.63.84} {43.63}.

FIGS. 6A-6D illustrate the sensing properties of Bi(HHTP). FIGS. 6A-6B illustrate the chemiresistive responses of devices with integrated Bi(HHTP) under an applied voltage of 1.0 V and an atmosphere of dry nitrogen to 15 minutes of exposure to NH₃ (FIG. 6A) and NO (FIG. 6B) at 40, 20, 10 and 5 ppm. FIG. 6C provides a plot of concentration vs normalized change in conductance (−ΔG/Gₒ). FIG. 6D provides the initial rate of response as a function of concentration during the first minute of exposure across two gases (NO and NH₃). Error bars represent standard deviations from the mean after averaging from 3 devices.

FIGS. 7A-7B illustrate the additional sensing properties of Bi(HHTP). FIG. 7A illustrates the chemiresistive response of Bi(HHTP) to varying concentrations of acetone (upper panel) and ethanol (lower panel) collected at temperatures 25-45° C. FIG. 7B shows a plot of concentration vs normalized change in conductance (−ΔG/Gₒ) for exposures to acetone and ethanol collected through variation in temperature of vapor generator.

FIGS. 8A-8D show the investigation of sensing mechanisms using Fourier-Transform Infrared spectroscopy of pristine and NH₃-doped Bi(HHTP) (FIG. 8A), and pristine and NO-doped Bi(HHTP) (FIG. 8B). FIG. 8C shows the X-ray photoelectron spectroscopy of the Bi4f_{7/2} and the Bi4f_{5/2} region of pristine Bi(HHTP) and NH₃-doped Bi(HHTP). FIG. 8D shows the X-ray photoelectron spectroscopy of the Bi4f_{7/2} and the Bi4f_{5/2} regions of pristine Bi(HHTP) and NO-doped Bi(HHTP).

FIGS. 9A-9F illustrate the incorporation of Bi(HHTP) into textiles and the characterization of the textiles. FIG. 9A illustrates the hydrothermal self-assembly of Bi(HHTP) SOFT e-textiles.

FIGS. 9B-9D show a Bi(HHTP) SOFT Cotton swatch (FIG. 9B) and a corresponding SEM image (FIG. 9C) to display Bi(HHTP) growth on threads (FIG. 9D) for the eventual development of Bi(HHTP) SOFT radiation detecting devices. FIG. 9E shows a space filling diagram of Bi(HHTP)'s herringbone-like structure. FIG. 9F summarizes Bi(HHTP) SOFT mass deposition and surface resistivity results.

FIGS. 10A-10C provide data relating to the characterization of Bi(HHTP)-containing textiles. FIG. 10A shows powder X-Ray diffractometry scans of Bi(HHTP) SOFT scuba and cotton devices compared with simulated Bi(HHTP) and black cotton and scuba textiles with peak matching for the (002), (200) and (202) planes. FIGS. 10B-10C show SEM micrographs of Bi(HHTP) SOFT scuba (FIG. 10B) and cotton (FIG. 10C) samples at the multifiber and single fiber scale down to a focused ion beam image (FIB).

FIGS. 11A-11B show ATR-IR Analysis of Bi(HHTP) on scuba (FIG. 11A) and cotton (FIG. 11B) textiles with highlighted changes in band structures that illustrate chemisorptive interactions between the coordination network and the textile substrate.

FIGS. 12A-12D provide illustrations and data related to radiation attenuation capabilities of Bi(HHTP).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Crystalline conductive coordination polymers (CPs), such as metal-organic frameworks (MOFs) and coordination networks (CNs), offer a promising alternative as a new emerging class of materials with broad applicability in chemiresistive detection. High conductivity, tunable surface chemistry, combined with modular porosity and high surface area for gas uptake-all accessible through bottom up self-assembly-give this class of materials a set of unique attributes that are particularly well suited for applications in, for example, gas sensing or radiation absorption. Despite this promise, most conductive coordination polymers that have thus far been employed in chemical sensing have two significant shortcomings. First, they are based on two-dimensional (2D) lattices having first row transition metals with square planar or octahedral coordination geometries around the metal site. While these low-dimensional materials exhibit high sensitivity to small reactive gases and vapors, the reliance on 2D lattices fundamentally limits gains in selectivity that can be achieved through stereoelectronic tuning of a binding site with a more complex coordination geometry. To address this fundamental limitation, it was reasoned that expanding beyond first row transition metals to create conductive networks with complex topologies and new, unsaturated coordination environments may promote gains in selectivity by simultaneous tuning of steric and electronic attributes of intermolecular interactions of sensing materials with analytes.

Second, integrating unsaturated coordination sites based on strong coordination bonds capable of favorable charge delocalization can provide room to investigate the contributions of structural features in relation to sensing within a well-ordered material. Furthermore, due to the challenges with obtaining suitably large crystallites of 2D framework materials, the lack of single crystal diffraction studies in established 2D systems conceals structural information on the surface and edge sides of the framework materials. This limitation restricts the fundamental understanding of the interactions of host frameworks materials with guest analytes.

Accordingly, a need exists for more effective crystalline materials for numerous applications. Various embodiments of the present disclosure address the aforementioned need.

In some embodiments, the present disclosure pertains to compositions that include a metal-organic framework with a plurality of metals and a plurality of ligands coordinated with the plurality of metals.

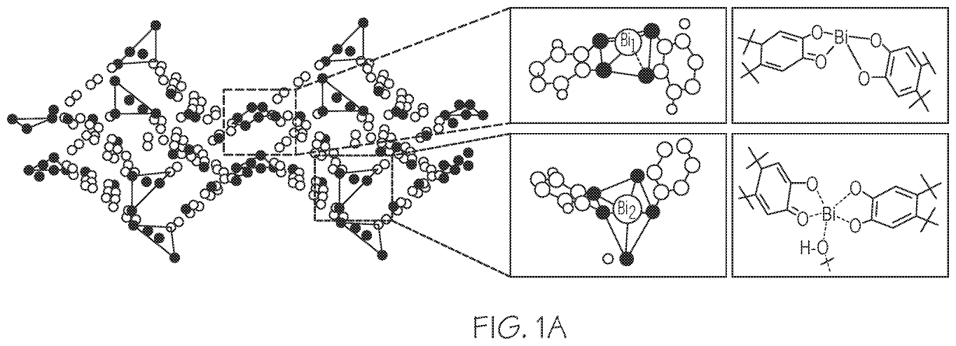

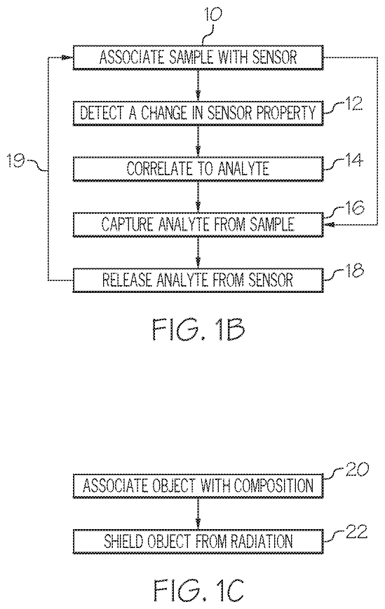

In some embodiments, the plurality of metals of the metal-organic framework include bismuth. In some embodiments, the plurality of ligands include a plurality of hydroxy moieties that are not part of carboxyl groups. In some embodiments, at least some of the hydroxy moieties are coordinated with bismuth to form Bi—O bonds. In some embodiments, the metal-organic framework is in the form of a conductive and interconnected network. A non-limiting example of a metal-organic framework of the present disclosure is illustrated in FIG. 1A.

In additional embodiments, the present disclosure pertains to methods of detecting an analyte in a sample. In some embodiments illustrated in FIG. 1B, the methods of the present disclosure include one or more steps of: associating the sample with a composition of the present disclosure that is in the form of a sensor (step 10); and detecting the presence or absence of the analyte from the sample through detecting a change in a property of the sensor (step 12) and correlating the change in the property to the presence or absence of the analyte (step 14). In some embodiments, the methods of the present disclosure also include the capture of the analyte from the sample by the sensor (step 16). In some embodiments, the capture and sensing of the analyte occurs simultaneously.

In some embodiments, the analyte detection methods of the present disclosure also include a step of releasing analytes from the sensor (step 18). In some embodiments, the sensor may then be reused to detect more analytes (step 19).

Additional embodiments of the present disclosure pertain to methods of shielding an object from radiation. In some embodiments illustrated in FIG. 1C, the radiation shielding methods of the present disclosure include a step of associating the object with a composition of the present disclosure (step 20) in order to shield the object from radiation (step 22).

Further embodiments of the present disclosure pertain to methods of making the metal-organic frameworks of the present disclosure by associating a plurality of metals and a plurality of ligands to result in the coordination of the plurality of metals with the plurality of ligands.

As set forth in more detail herein, the methods and compositions of the present disclosure can have numerous embodiments. For instance, the metal-organic frameworks in the compositions of the present disclosure can include various metals and ligands in various arrangements. Moreover, the compositions of the present disclosure can be utilized in numerous manners to form, for example, sensors, dosimeters, and textiles. Additionally, the compositions of the present disclosure can have various forms and be associated with various polymers.

Furthermore, various methods may be utilized to sense analytes using the compositions of the present disclosure. For example, various methods may be utilized to detect various changes in various properties of the sensors of the present disclosure and correlate the changes in properties to the presence or absence of various analytes.

Moreover, various methods may be utilized to shield various objects from various sources of radiation by associating the objects with various compositions of the present disclosure. Additionally, various methods may be utilized to make the compositions of the present disclosure.

### Compositions

As set forth in more detail herein, the compositions of the present disclosure generally include metal-organic frameworks with various metals and ligands in various forms. Additionally, the metal-organic frameworks may be associated with various components, such as various sensors, textiles, dosimeters, and polymers.

### Metals

The metal-organic frameworks of the present disclosure can include various types of metals. For instance, in some embodiments, the plurality of metals can include bismuth. In some embodiments, the bismuth is in the form of six-coordinate bismuth.

In some embodiments, the metal-organic frameworks can further include one or more addition metals. In some embodiments, the additional metals can include, without limitation, divalent metals, transition metals, iron, nickel, copper, cobalt, zinc, manganese, platinum, palladium, gold, chromium, magnesium, tin, and combinations thereof.

In some embodiments, the plurality of metals includes essentially bismuth. In some embodiments, the plurality of metals excludes metals other than bismuth.

### Ligands

The metal-organic frameworks of the present disclosure can include various types of ligands. For instance, in some embodiments, the plurality of ligands can include, without limitation, organic ligands, hexatopic ligands, aromatic ligands, phthalocyanine-based ligands, metallophthalocyaline-based ligands, naphthalocyanine-based ligands, polydentate ligands, bidentate ligands, tridentate ligands, triphenylene-based ligands, triphenylene derivatives, hexahydroxytriphenylene-based organic linkers, hexaiminotriphenlyene-based organic linkers, hexahydroxybenzene-type ligands, hexahydroxytrinaphthalene ligands, thiol-based ligands, hydroxy-based ligands, and combinations thereof.

In some embodiments, the plurality of ligands include hydroxy-based ligands. In some embodiments, the hydroxy-based ligands include aromatic ligands. In some embodiments, the hydroxy-based ligands include a plurality of hydroxy moieties. In some embodiments, the hydroxy moieties are not part of carboxyl groups. In some embodiments, at least some of the hydroxy moieties are coordinated with bismuth to form Bi—O bonds. In some embodiments, at least some of the hydroxy moieties are coordinated with bismuth to form Bi—O bonds. In some embodiments, at least some of the hydroxy moieties remain free and uncoordinated. In some embodiments, at least some of the hydroxy moieties form hydrogen bonds with water molecules. In some embodiments, the hydroxy moieties that coordinate with bismuth to form Bi—O bonds are not part of carboxyl groups.

In some embodiments, the plurality of ligands include triphenylene-based ligands. In some embodiments, the triphenylene-based ligands can include, without limitation, 2,3,5,6,10,11-hexahydroxytriphenylene (HHTP), 2,3,5,6,10,11-hexaiminotriphenylene (HITP), 2,3,5,6,10,11-hexathiotriphenylene (HTTP), and combinations thereof. In some embodiments, the plurality of ligands includes 2,3,5,6,10,11-hexahydroxytriphenylene (HHTP).

In some embodiments, the metal-organic framework has the following formula of M_{y}(HXTP)_{z}. In some embodiments, M includes bismuth. In some embodiments, X is O or NH. In some embodiments, y is 1 or 3. In some embodiments, z is 1 or 2. In some embodiments, HXTP represents a triphenylene-based ligand that can include, without limitation, 2,3,5,6,10,11-hexahydroxytriphenylene (HHTP), 2,3,5,6,10,11 hexaiminotriphenylene (HITP), 2,3,5,6,10,11-hexathiotriphenylene (HTTP), and combinations thereof.

In some embodiments, the metal-organic framework has the following formula of M₃(HXTP)₂. In some embodiments, M is bismuth. In some embodiments, X is O or NH. In some embodiments, HXTP represents a triphenylene-based ligand that can include, without limitation, 2,3,5,6,10,11-hexahydroxytriphenylene (HHTP), 2,3,5,6,10,11-hexaiminotriphenylene (HITP), 2,3,5,6,10,11-hexathiotriphenylene (HTTP), and combinations thereof. In some embodiments, the metal-organic framework is Bi₃(HHTP)₂.

In some embodiments, the metal-organic framework has the following formula of M(HXTP). In some embodiments, M is bismuth. In some embodiments, the metal-organic framework is Bi(HHTP).

In some embodiments, the metal-organic framework is Bi(HHTP)-α. In some embodiments, Bi(HHTP)-α has a 2,3-c nodal net stoichiometry.

In some embodiments, the metal-organic framework is Bi(HHTP)-β. In some embodiments, Bi(HHTP)-β has a 3,4,4,5-c nodal net stoichiometry.

### Sensors

In some embodiments, the metal-organic frameworks of the present disclosure are a component of a sensor. In some embodiments, the metal-organic frameworks are associated with a sensor surface. In some embodiments, the metal-organic frameworks serve as a sensor surface. In some embodiments, the sensor surface can include, without limitation, a conductive substrate, a carbon-based substrate, glassy carbon, and combinations thereof.

In some embodiments, the sensor can include, without limitation, electrodes, carbon electrodes, glassy carbon electrodes, gold electrodes, solid contact electrodes, and combinations thereof. In some embodiments, the sensor further includes a wiring and a potentiostat. In some embodiments, the wiring electrically connects the sensor surface to the potentiostat. In some embodiments, the sensor further includes an output display. In some embodiments, the output display is electrically connected to the potentiostat.

Additionally, in some embodiments, the sensor is in the form of a sensing array. In some embodiments, the sensor is in the form of a portable sensor. In other embodiments, the metal-organic framework is a reversible sensor.

### Dosimeters

In some embodiments, the metal-organic frameworks of the present disclosure are a component of a dosimeter. In some embodiments, the metal-organic frameworks are associated with the dosimeter. In some embodiments, the metal-organic frameworks constitute a sensing component of the dosimeter.

### Textiles

In some embodiments, the metal-organic frameworks of the present disclosure are associated with a textile. In some embodiments, the textile includes a plurality of fibers and a plurality of pores. In some embodiments, the metal-organic frameworks are associated with the fibers of the textile. In some embodiments, the metal-organic frameworks are dispersed within the fibers of the textile.

The metal-organic frameworks of the present disclosure may be associated with various textiles. For instance, in some embodiments, the textiles can include, without limitation, synthetic textiles, polyester based textiles, poly-batting based textiles, natural textiles, cotton-based textiles, wool, fabrics, fabric swatches, cotton, commercial textiles, natural woven cotton fabric, non-woven polyester batting, and combinations thereof.

### Personal Protective Equipment

In some embodiments, the metal organic frameworks of the present disclosure are a component of personal protective equipment. As such, in some embodiments, the metal-organic frameworks of the present disclosure can be associated with a personal protective equipment. In some embodiments, the personal protective equipment is a radiation protecting garment, such as a radiation protection suit.

### Metal-Organic Framework Structures

The metal-organic frameworks of the present disclosure can have various structures. For instance, in some embodiments, the metal-organic frameworks include a non-planar shape. In some embodiments, the non-planar shape includes a three-dimensional shape. In some embodiments, the non-planar shape includes a polyhedral shape. In some embodiments, the polyhedral shape includes a tetragonal pyramid shape. In some embodiments, the polyhedral shape includes a quadrilateral shape.

In some embodiments, the metal-organic frameworks of the present disclosure have a polyhedral shape. In some embodiments, the polyhedral shape is defined by a plurality of ligands coordinated with bismuth through Bi—O bonds. In some embodiments, the Bi—O bonds are derived from the bonding of bismuth with the hydroxy moieties of the ligands. In some embodiments, the hydroxy moieties are not part of carboxyl groups.

In some embodiments, the metal-organic frameworks of the present disclosure are in the form of a layer, a powder, a compressed powder, a pellet, a pencil-lead, a free-standing or substrate-supported wire, a free-standing or substrate-supported array of wires, a free-standing film, a substrate-supported film, and combinations thereof. In some embodiments, the metal-organic frameworks are in the form of nanosheets, nanorods, and combinations thereof. In some embodiments, the metal-organic frameworks are in the form of a layer.

In some embodiments, the metal-organic frameworks of the present disclosure include a non-planar shape that includes a layered shape. In some embodiments, the layered shape is defined by a plurality of interconnected layers that are interconnected through Bi—O bonds. In some embodiments, the Bi—O bonds arise from the coordination of bismuth with hydroxy groups of metal-organic framework ligands. In some embodiments, the hydroxy groups are not part of carboxyl groups.

In some embodiments, the metal-organic frameworks of the present disclosure include a crystalline shape. In some embodiments, the crystalline shape includes rectangular-shaped crystallites.

The metal-organic frameworks of the present disclosure may have various surface areas. For instance, in some embodiments, the metal-organic frameworks of the present disclosure have a surface area of at least about 20 m² g⁻¹. In some embodiments, the metal-organic frameworks of the present disclosure have a surface area of at least about 25 m² g⁻¹.

The metal-organic frameworks of the present disclosure may have various porosities. For instance, in some embodiments, the metal-organic frameworks of the present disclosure have small pores.

### Polymers

The metal-organic frameworks of the present disclosure can include various polymers or be in various polymeric forms. For instance, in some embodiments, the metal-organic framework is associated with a polymer. In some embodiments, the metal-organic framework is in the form of a polymer composite. In some embodiments, the polymer can include, without limitation, fluoropolymers, polytetrafluoroethylene, Nafion, polymer matrices, and combinations thereof. In some embodiments, the metal-organic framework is dispersed within the polymer.

### Methods of Sensing Analytes

As set forth previously, the analyte sensing methods of the present disclosure include one or more of the following steps: (1) associating a sample with a composition of the present disclosure that is in the form of a sensor; and (2) detecting the presence or absence of an analyte from the sample by (a) detecting a change in a property of the sensor, and (b) correlating the change in the property to the presence or absence of the analyte. As set forth in more detail herein, the compositions of the present disclosure can be utilized to sense various analytes in various manners.

### Changes in Properties

The methods of the present disclosure can detect various changes in properties of the sensors of the present disclosure. For instance, in some embodiments, the change in the property of the sensor includes a change in normalized conductance over time (ΔG/Go).

### Correlating

The methods of the present disclosure can correlate changes in properties of sensors to the presence or absence of analytes in various manners. For instance, in some embodiments, the correlating includes comparing the properties of the sensor to properties of the sensor in response to association with known analytes. In some embodiments, the comparing occurs by comparing the properties of the sensor to a database that includes properties of sensors associated with known analytes.

### Samples

The methods of the present disclosure can detect analytes in various types of samples. For instance, in some embodiments, the sample is derived from a gaseous environment. In some embodiments, the sample is derived from a solution. In some embodiments, the sample is a gas. In some embodiments, the sample is a liquid. In some embodiments, the sample is in the form of a gas and a liquid.

### Analytes

The methods of the present disclosure can detect the presence or absence of various analytes in a sample. For instance, in some embodiments, the analyte can include, without limitation, gases, ketones, alcohols, aromatic compounds, volatile organic compounds, water, neurotransmitters, hormones, proteins, sugars, metal ions, ionizing radiation, NO, CO, H₂S, NH₃, H₂O, and combinations thereof.

In some embodiments, the analyte includes one or more gases. In some embodiments, the one or more gases can include, without limitation, NO, CO, H₂S, NH₃, H₂O, and combinations thereof.

In some embodiments, the analyte includes one or more volatile organic compounds. In some embodiments, the one or more volatile organic compounds can include, without limitation, acetone, methanol, ethanol, isopropanol, and combinations thereof.

In some embodiments, the analyte includes one or more ionizing radiations. In some embodiments, the one or more ionizing radiations can include, without limitation, X-rays, ultraviolet (UV)-rays, and combinations thereof.

In some embodiments, the method is utilized for the simultaneous detection of a plurality of different analytes. In some embodiments, the method is utilized to quantify the concentration of the analyte.

### Exposing

The methods of the present disclosure can also utilize various methods to expose a sample to a sensor. For instance, in some embodiments, the exposing includes flowing the sample through the sensors of the present disclosure. In some embodiments, the exposing includes incubating the sample with the sensor. In some embodiments, the exposing results in the reversible association of analytes in the sample with the composition. In some embodiments, the association also results in at least one of filtration, pre-concentration, decontamination, and capture of the analyte by the sensors of the present disclosure.

In some embodiments, the association also results in the capture of the analyte by the sensor. As such, in some embodiments, the methods of the present disclosure may be utilized to simultaneously detect and capture analytes from various samples. In more specific embodiments, the methods of the present disclosure may be utilized to simultaneously detect and capture ionizing radiation from various samples.

### Detection

The methods of the present disclosure can detect analytes, or multiple analytes, at various concentrations. For instance, in some embodiments, the analytes are detectable at concentrations of less than about 100 ppm. In some embodiments, the analytes are detectable at concentrations of less than about 10 ppm. In some embodiments, the analytes are detectable at concentrations of less than about 1 ppm. In some embodiments, the analytes are detectable at concentrations of less than about 0.5 ppm. In some embodiments, the analytes are detectable at concentrations of less than about 0.4 ppm. In some embodiments, the analytes are detectable at concentrations that range from about 10 nM to about 100 μM. In some embodiments, the analytes are detectable at concentrations that range from about 10 nM to about 100 nM.

### Release

The methods of the present disclosure can also include additional steps. For instance, in some embodiments, the methods of sensing analytes further include a step of releasing the analyte from the sensor. In some embodiments, the releasing occurs by washing the sensor.

Methods of Shielding an Object from Radiation

Additional embodiments of the present disclosure pertain to methods of shielding objects from radiation. Such methods generally include associating the object with a composition of the present disclosure.

### Objects

The methods of the present disclosure can shield radiation from various objects. For instance, in some embodiments, the object can include, without limitation, an aircraft, a spacecraft, a space station, a human being, an animal, and combinations thereof. In some embodiments, the object is a human being.

### Associating

The methods of the present disclosure can utilize various methods for associating the compositions of the present disclosure with the objects. For instance, in some embodiments, the associating occurs by a method that can include, without limitation, wrapping, adhering, surrounding, wearing, and combinations thereof. In more specific embodiments, the compositions of the present disclosure are in the form of a textile that is wearable by a human being to shield the human being from radiation. In some embodiments, the object is a human being, the composition is in the form of a personal protective equipment, and the associating occurs by wearing the personal protective equipment.

### Shielding

The methods of the present disclosure can shield objects from radiation by various methods. For instance, in some embodiments, the shielding occurs by absorption of the radiation by the composition.

### Radiation

The methods of the present disclosure can shield objects from various types of radiation. For instance, in some embodiments, the radiation is ionizing radiation. In some embodiments, the radiation is from X-rays. In some embodiments, the radiation is from ultraviolet (UV) rays.

### Methods of Making Metal-Organic Frameworks

Additional embodiments of the present disclosure pertain to methods of making the compositions of the present disclosure. Such methods generally include one or more of the following steps of associating a plurality of metals, such as those discussed in the Application, and a plurality of ligands, such as those discussed in the Application. In some embodiments, the plurality of metals includes bismuth. In some embodiments, the associating results in the coordination of the plurality of metals with the plurality of ligands to form the metal-organic frameworks of the present disclosure.

### Associating

The methods of the present disclosure can utilize various methods of association, and can be associated in the presence of an object. For instance, in some embodiments, the association occurs in situ in the presence of one or more objects. In some embodiments, the metal-organic frameworks form in situ on a surface of the one or more objects. In some embodiments, the one or more objects can include, but are not limited to, polymers (such as the polymers described in the Application), textiles (such as the textiles described in the Application), sensors (such as the sensors described in the Application), dosimeters (such as the dosimeters described in the Application), and combinations thereof.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Conductive Stimuli-Responsive Coordination Network Linked with Bismuth for Chemiresistive Gas Sensing

This Example describes the design, synthesis, characterization, and performance of a novel semiconductive crystalline coordination network, synthesized using 2,3,6,7,10,11-hexahydroxytriphenylene (HHTP) ligands interconnected with bismuth ions, towards chemiresistive gas sensing. Bi(HHTP) exhibited two distinct structures upon hydration and dehydration of the pores within the network, Bi(HHTP)-α and Bi(HHTP)-β, respectively, both with unprecedented network topology (2,3-c and 3,4,4,5-c nodal net stoichiometry, respectively) and unique corrugated coordination geometries of HHTP molecules held together by Bi ions, as revealed by microelectron diffraction with atomic resolution. Good electrical conductivity (5.3×10⁻³ S·cm⁻¹) promotes the utility of this material in chemical sensing of gases (NH₃ and NO) and volatile organic compounds (VOCs). The chemiresistive sensing of NO and NH₃ using Bi(HHTP) display limits of detection of 0.15 ppm and 0.29 ppm, respectively, at low driving voltages (0.1-1.0 V) and operation at room temperature. Spectroscopic assessment via X-ray photoelectron spectroscopy (XPS) and Fourier-transform infrared spectroscopy (FT-IR) suggested that the sensing mechanisms involved in the sensing response of Bi(HHTP) to gaseous analytes is accompanied by charge transfer interactions.

In this Example, Applicants show that Bi(HHTP), whose flexible structure was determined via microcrystal electron diffraction techniques, exhibits electrical conductivity, new topology (2,3-c and 3,4,4,5-c nodal net stoichiometry), and high sensitivity towards specific gases and vapors. Applicants chose a representative class of oxidative and reducing gaseous probes and volatile organic compounds (VOCs) with varying polarities and steric sizes to examine the effects of these parameters on sensing response of Bi(HHTP). Specifically, Applicants demonstrate that Bi(HHTP) exhibits unique chemiresistive responses to ppm-concentrations of NH₃ (LOD of 0.29 ppm) and NO (LOD of 0.15 ppm) and four VOCs (acetone, ethanol, methanol and isopropanol). The response to NO is characterized by substantial reversibility, which is notable as many crystalline conductive coordination polymers exhibit dosimetric responses towards this oxidizing gas. The chemiresistive response towards ammonia is characterized by decreased electrical resistance and partial reversibility at ppm concentrations. The combined use of several spectroscopic techniques, such as X-ray photoelectron spectroscopy (XPS), attenuated total reflection infrared spectroscopy (ATR-IR), and electron paramagnetic resonance (EPR), provided insight into the mechanism of sensing and host-guest sites within Bi(HHTP). Applicants demonstrate that the sensing mechanism towards NH₃ and NO involves redox interactions promoted by gaseous analytes adsorbed on the surface of the conductive coordination network.

### Example 1.1. Molecular Design

The molecular design of the conductive coordination network capitalizes on several unique characteristics of Bi-containing compounds and materials and the molecular design extends these characteristics to generate a new material with promising functionality. Currently, bismuth-based materials and coordination compounds have applications in healthcare, photocatalytic function, radiation technology, and gas adsorption and storage. The unique flexible coordination sphere of bismuth, Lewis acidity, non-toxicity, stability, as well as the high affinity for soft and hard ligands of Bi ions enable desirable structure-property relationships, particularly when bismuth is used as a constituent within coordination polymers (CPs) or hybrid networks comprised of metal nodes linked by organic ligands. Specifically, bismuth-based CPs and porous metal-organic frameworks (MOFs) have demonstrated interesting structure-property relationships such as conductivity, photocatalysis, and photoluminescence. These properties are tunable through the strategic selection of constituent organic linkers in bismuth containing CPs that can dictate the coordination environment around the Bi metal node.

The unique nature of bismuth-based coordination networks allows for the tailoring of multiple useful functions, such as charge delocalization, band gap, and direction of assembly, or dimensionality properties, through careful selection of the organic ligands. Several of these properties are highly desirable in the context of chemiresistive sensing. First, conductive CPs may be designed by selecting constituents which contain loosely held valence shell electrons and ligands that permit their efficient through-bond charge delocalization, allowing the integration of the semiconductive material into amperometric devices for chemical sensing. This charge delocalization has been demonstrated within both bismuth oxide lattices and bismuth-based metal-organic coordination networks.

Second, the flexible coordination geometry of bismuth provides control over dimensionality of the coordination network structure, resulting in unique structure-property relationships through ligand modification strategies and through the choice of bismuth metal salt. Third, the structures of Bi(III) containing compounds often present a vacant coordination site at the Bi center, which may serve as an electron acceptor site. This coordinatively unsaturated environment around bismuth may undergo further interaction with analytes, thereby enabling selective chemical detection of analytes with a 3D coordination sphere of Bi accompanied by electronic transduction of signal. Capitalizing on these advantages can provide a path to control functional properties for use in selective chemical sensing.

Applicants' molecular design is inspired by previously reported literature of bismuth-based semiconductive coordination networks interconnected with triphenylene-based ligands. Hexakis(alkylthio)triphenylene (alkyl: methyl, ethyl, and isopropyl) triphenylenes reacted with bismuth halides to produce semiconductive hybrid networks that featured flexible network dimensionalities and electronic properties. Applicants reasoned that substituting the alkyl-thio substituents with hydroxy groups may promote similar coordination chemistry with Bi, while generating a material with good stability to water and air due to the robust nature of hydroxy-substituted triphenylenes and the strong nature of Bi—O bonds, compared to their sulfur substituted analogs.

2,3,6,7,10,11-hexahydroxytriphenylene (HHTP) exhibits a large π-conjugated system and three-fold symmetry and has previously been reported to form conductive metal-organic frameworks using first row transition metals and lanthanides. A unique attribute of HHTP and HHTP-based MOFs is that they are capable of undergoing electron transfer interactions that can be coupled to proton transfer events. This coupling may provide an additional level of selectivity in sensing devices for proton containing guests. However, CPs of this ligand are unprecedented for metal complexes with bismuth.

Accordingly, Applicants aimed to achieve bottom-up assembly of a conductive coordination polymer that provides a 3-dimensional (3D) ligand coordination environment around the metal center tailored for enhanced selectivity in response to specific gaseous molecules. Applicants subjected Bi(III) acetate to aqueous reaction conditions to observe the structure-property relationships this metal node will possess when paired with a polyaromatic organic linker. Applicants observed that Bi(HHTP) exhibits distinct structural transformations upon hydration and dehydration of the pores within the network, likely driven by hydrogen bonding with the oxo groups on the ligand, which induced changes in coordination environment of both Bi centers and unit cell parameters. This type of dynamic flexibility, such as the slipping and/or expansion of the layers, has been investigated in 2D HHTP-based MOFs using quantum mechanical calculations,⁶³ along with the higher propensity for open metal sites in Co²⁺ to accept water coordination over Cu²⁺.

### Example 1.2. Synthesis and Characterization

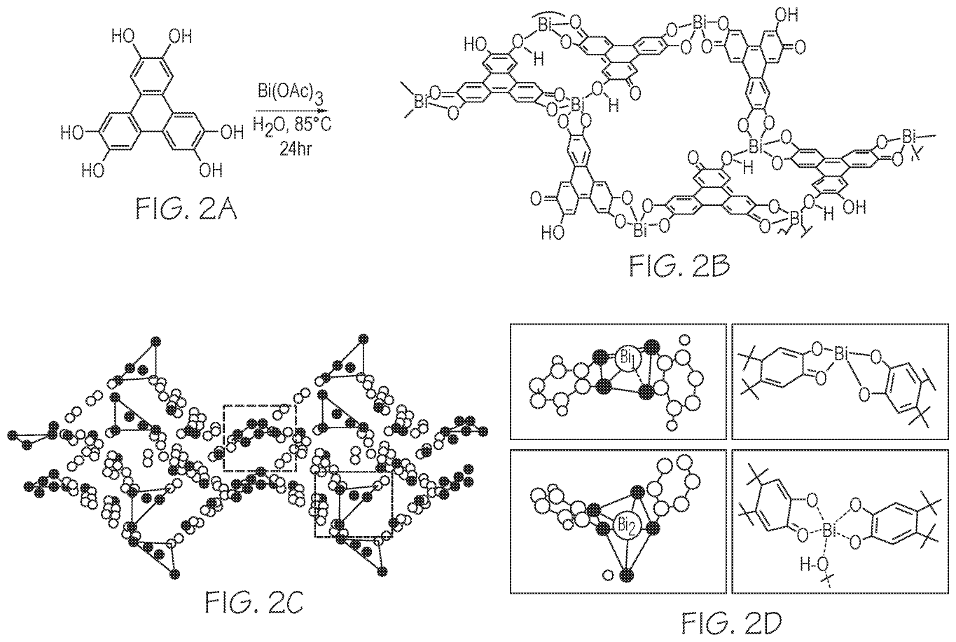

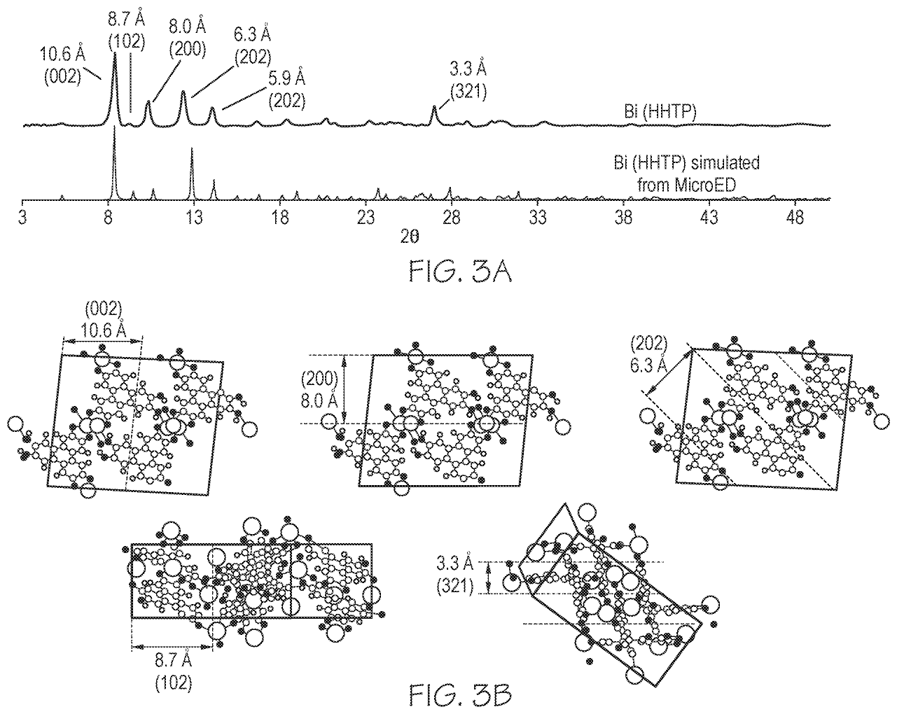

Applicants used hydrothermal synthesis that combined Bi(OAc)₃ and HHTP to produce Bi(HHTP) (FIGS. 2A-2D). Reaction optimization procedures were carried out after powder X-ray diffractometry (pXRD) analysis revealed the presence of residual starting material Bi(OAc)₃ when Bi(HHTP) was synthesized using a 2:1 molar ratio of HHTP and Bi(OAc)₃. This residual starting material can be removed with either a water purification procedure (overnight stirring in H₂O at 50° C.) followed by subsequent ethyl acetate washes or a Soxhlet extraction technique using the organic solvent ethyl acetate. The resulting dark green/blue conductive, microcrystalline powder [Bi(HHTP)], was initially characterized using pXRD analysis (FIGS. 3A-3B), scanning and transmission electron microscopy, (SEM and TEM, respectively), and elemental analysis. The pXRD pattern of Bi(HHTP) exhibited a high-intensity peak in the low angle range at 8.36° 2θ. This peak corresponds to an interatomic distance of 10.6 Å and the (002) plane, which bisects the unit cell of Bi(HHTP) (FIGS. 3A-3B). Other major peaks appearing in the pXRD pattern included the (102̅), (200), (202̅), (202) and (321̅) planes which were attributed to interatomic distances of 8.7 Å, 8.0 Å, 6.3 Å, 5.9 Å and 3.3 Å using Bragg law, respectively. The (002) and (202̅) planes intersect a section of the HHTP ligand when viewing along the crystallographic c-axis, while the (200) and the (102̅) planes intersect and run parallel to the Bi atoms (FIG. 3B). The (321̅) crystalline plane runs parallel to the π-π stacking distance and corresponds to an interatomic distance of 3.3 Å.

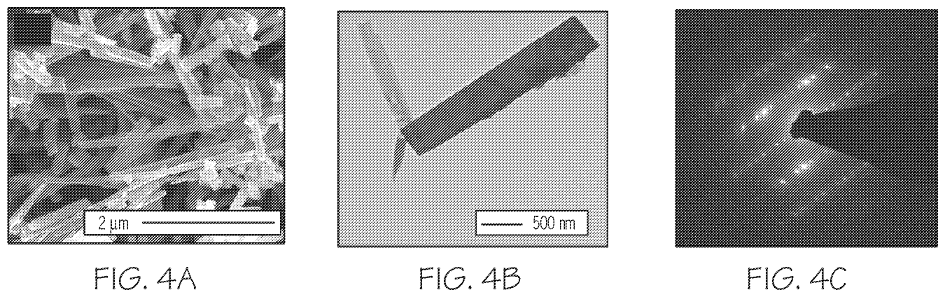

Morphological characterization of Bi(HHTP) via SEM analysis revealed rectangular-shaped crystallites of varying lengths (FIG. 4A). TEM imaging, obtained after 1.0 mg of Bi(HHTP) was sonicated in acetone for 16 hours and drop-casted onto a carbon grids provided visualization of rectangular, sheet-like materials. Further characterization of Bi(HHTP) using TEM analysis (exfoliated by overnight sonication) revealed the presence of a distinct crystallite with a length of ˜2-μm (FIG. 4B). Selected area diffraction analysis (SAED) on this crystallite material showed bright, well-ordered diffraction spots in reciprocal space (FIG. 4C), corresponding to observed interatomic distances. The distances between the diffraction spots were calculated according to the equation derived from Bragg's law. Two interatomic distances (3.3 Å ad 6.3 Å) measured within the SAED nanocrystal are also present in the pXRD pattern (FIGS. 3A-3B). The 6.3 Å distance observed in the nanocrystal is slightly offset in the pXRD (6.6 Å) and likely corresponds to the (202̅) hkl plane, while the 3.3 Å interatomic distance reflects the (321̅) plane, which runs parallel to the π-π stacking distances (FIG. 3B).

Due to the crystal size within the bulk powder, single-crystal X-ray diffraction (SCXRD) was unsuccessful. Thus, Applicants focused on microelectron diffraction (MicroED). Although the MicroED method was popularized by structural biologists for the characterization of proteins, the technique has proven invaluable for the field of small molecule characterization, and even more recently, the determination of both coordination networks and MOFs. This method enabled the structural characterization of Bi(HHTP) and permitted the correlation of the hkl planes in this structure to the ones observed in the experimental pXRD spectrum (FIGS. 3A-3B).

### Example 1.3. Crystallographic Analysis of Structure from MicroED

For MicroED analysis, electron diffraction data was collected using a Talos F200C transmission electron microscope equipped with a Thermo-Fischer CetaD detector. To prepare the sample grids (Quantifoil or pure Carbon TEM grids), a TEM grid was placed in a vial containing dry powder and gently shaken. Images were collected in a movie format as crystals were continuously rotated under a focused electron beam. Typical data collection was performed using a constant tilt rate of 0.3°/s between the minimum and maximum tilt ranges of −72° to +72°, respectively. Structural characterization by MicroED uncovered that Bi(HHTP) exhibited two polymorphs, Bi(HHTP)-α and Bi(HHTP)-β. Bi(HHTP)-α exhibited monoclinic (α, γ=90°, β=94°) type Bravais lattice with symmetry group P 2₁/c and intricately connected layers (vide infra). Bi(HHTP)-β displayed different cell parameters (α, γ=90°, β=97°), occupied pores (likely water molecules from incomplete drying) and distinct coordination geometries, but the same symmetry group, P 2₁/c. Pawley refinement was conducted using the crystallographic information file (cif.) obtained from MicroED, determining the unit cell parameters and the calculation presented a R_{wp} of 7.07% and an Rₚ of 12.54%.

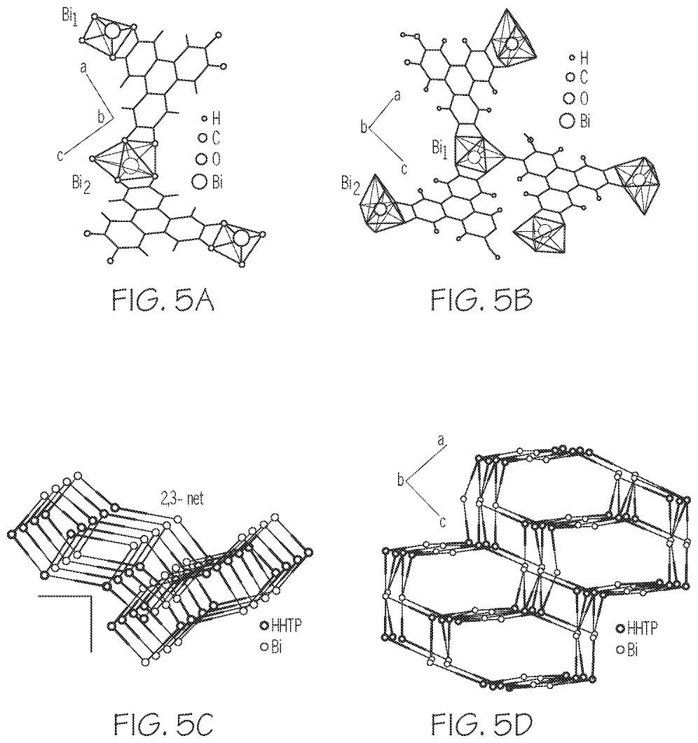

Topological analysis performed using ToposPro program package and Topological Types Database (TTD) collection of periodic networks was used to reveal the network topology model in the coordination network. The topological description includes a simplification procedure (graph theory approach), which was used to describe the crystal net topology and designate a 2,3-C4 topological type net for Bi(HHTP)-α, which corresponds to this structure in its standard representation (FIG. 5C). The cluster simplification procedure was also implemented to identify more complex building units of a structure and characterize their connection mode, where fragments Bi(HHTP)-α form infinite chains that are linked together through an oxygen atom and exhibit rod packing with 2M4-1 topology and point symbol.

The Bi(HHTP)-α sheet contains dimeric 1D zigzagging chains of alternating nonplanar HHTP ligands that connect one 1D chain to another through the longest Bi—O bond of 2.6 Å. These dimeric chains contain alternating uncoordinated semiquinone groups and are stacking in the crystallographic b-direction through π-π stacking interactions. Each Bi—Bi interaction present inside the one-dimensional chains connecting HHTP constituents is approximately 4.1 Å long. Both Bi(HHTP)-α and -β adopt a herringbone-like packing motif, similar to HHTP, where the Bi atoms cause distortions in the π-π stacking of the matrix through catechol bidentate chelation and slight rotation within the coordination sphere. Bi(HHTP)-α displays two coordination environments (FIG. 2D), one distorted tetragonal pyramid (Bi₂) and distorted quadrilateral (Bi₁), the former which is similar to a dimeric Bismuth(III) catecholate coordination complex involved in a five-coordination environment reported previously.

Bi(HHTP)-β exhibits two distinct Bi coordination spheres with 6-coordinate environments; one (Bi₂) containing an aqua ligand. Specifically, the coordination polyhedra of Bi₁ and Bi₂ as distorted pentagonal pyramid (CN=6) and distorted one-caped octahedron (CN=6), respectively. In this polymorph, the staircase-like one-dimensional chains are connected through the oxygen atoms on HHTP ligands to the 6-coordinate Bi₂ within a separate layer, closely resembling a three-dimensional network.

Without being bound by theory, Applicants hypothesize that polymorph Bi(HHTP)-β is stabilized when water occupies the slit-like pores of the network, altering the unit cell parameters and permitting further interaction of each oxygen heteroatom in HHTP to neighboring adjacent layers. The presence of uncoordinated hydroxy groups facing inward within the pores are likely further stabilized through hydrogen bonding with the aqua ligands within the pores of the network. Compared to Bi-based MOFs made using carboxylate ligands, which exhibit Bi—O bond lengths ranging from 2.2-3.0 Å, we observed a tighter range of bond lengths, 2.0-2.6 Å, in Bismuth catecholate coordination. The π-π stacking distance was measured to be 3.3 Å, which matches the interatomic distance obtained from diffraction peaks in pXRD.

### Example 1.4. Elemental Composition Analysis

Elemental microanalysis and inductively coupled plasma mass spectrometry (ICP-MS) confirmed the elemental composition of Bi(HHTP). The percent mass of carbon, hydrogen and bismuth observed experimentally within the coordination network (38.3%, 1.51% and 33.1%, respectively) were comparable to theoretical calculations (41.1%, 1.73% and 39.4%, respectively) based on the empirical formula (C₃₆H₁₂O₁₂)Bi₂. This calculation may also indicate the dominant distribution of Bi(HHTP)-α polymorph in the bulk material, based on the percentages of hydrogen in the elemental analysis indicating the absence of large amounts of water within the pores of the network which are present in Bi(HHTP)-β. Attenuated total reflectance infrared spectroscopy (ATR-IR) of Bi(HHTP) revealed the presence of vibrational frequencies at 1420 and 1157 cm⁻¹, which are characteristic of catechol-metal vibrations. The FT-IR spectrum of Bi(HHTP) also exhibited a significant reduction in relative magnitude of the hydroxy group band at 3400 cm⁻¹ relative to HHTP, which is evidence of a coordination event for the hydroxy moieties. Because the vibrational modes strongly depend on the atomic masses, the heavy Bi ions should present vibrational bands at lower frequencies (500-100 cm⁻¹). Thus, the appearance of new bands in this region may also be attributed to new Bi—O bond vibrational frequencies.

### Example 1.5. Oxidation State Analysis

X-ray photoelectron spectroscopy (XPS) enabled the analysis of Bi in low (3+) valence oxidation states through peaks at binding energies of 160.1 eV and 165.3 eV, assigned to Bi³⁺ 4f_{7/2} and Bi 4f_{5/2}. Applicants were unable to deconvolute the region of the oxygen is peak present at 532 eV to assign the C—O and C═O bonds, due to the likely presence if H₂O both within the pores of the network and within the coordination sphere of Bi(HHTP)-β creating uncertainty of the correct electronic state of the ligand. Based on the deconvoluted C is peaks and considering the presence of Bi(III), one possible oxidation state of the ligand to result in an overall neutral coordination network is a bis-semiquinone catechol state (sq, sq, cat) to generate Bi(III) within the network. The C is spectra is consistent with this oxidation state, as it presents C—O, C═O, and C—OH bonds in 2:2.6:1 ratio.

### Example 1.6. Surface Area Analysis

Structural characterization of the specific surface area of activated and degassed (at 85° C. and 635 Torr for 24 hours) Bi(HHTP) using Brauner-Emmet Teller (BET) analysis was performed using N₂ adsorption-desorption isotherms, collected at 77K on a Micromeritics 3FLEX instrument. Preliminary results indicated a surface area of 26.8 m² g⁻¹. The low surface area measured from BET analysis using nitrogen (probe radius of 1.8 Å) is reasonable when compared to the accessible solvent surface area calculated using Materials Studio© software, where a probe radius of 1.2 Å calculated a surface area 101.6 Å² and a free volume of 22.62 Å³ and a probe radius of 1.8 Å calculated a surface area of 0 Å².

### Example 1.7. Thermal Analysis

The thermal gravimetric analysis (TGA) profile of Bi(HHTP) revealed a total of ˜34% weight loss with the highest rate of decomposition occurring at 466° C. There was an initial mass loss of ˜8% from 100-200° C., potentially due to the loss of volatile solvent molecules such as acetone or H₂O. Applicants observed a similar mass loss for Bi(OAc)₃ (38%) and a higher mass loss for the organic linker, HHTP (56%).

### Example 1.8. Electronic Properties

Conductivity measurements of Bi(HHTP) were performed using a four-point probe technique, which required 100 mg of material pressed into 6 mm diameter pellet of 0.2 mm thickness. Bi(HHTP) showed a bulk conductivity of 5.3×10⁻³ S·cm⁻¹. Pellets of the precursors Bi(OAc)₃ and HHTP exhibited no measurable conductivity using a two-point probe digital multimeter (Extech EX430 series), which has a maximum resistance limit of measurement at 40 MΩ.

DFT calculations were performed on the simulated structure of Bi(HHTP) using a functional PBE and GGA approximations. The high symmetry points in the first Brillouin zone demonstrated that the Dirac bands approached the Fermi level through the Y-A and E-C (crystallographic c) directions where a low band gap of approximately 0.1 eV was observed for Bi(HHTP)-α and 0.08 eV for Bi(HHTP)-β. The partial density of states analysis showed that, compared with Bi, the p orbitals from the C and O atom contribute significantly to the Dirac bands. To investigate the Arrhenius activation energy for electrical conductivity of Bi(HHTP), a 2-point probe on a 50 mg pressed pellet was employed to collect the current change under different temperature (293 K to 383 K) with a linear sweep voltage from −2.0 V to 2.0 V. The activation energy determined by this method was 425 meV. The optical band gap was determined by plotting the absorbance squared vs energy (eV) and estimated to be 1.61 eV based on the value of absorption edge.

### Example 1.9. Chemiresistive Gas Sensing

Applicants hypothesized that Bi(HHTP) would be a promising chemiresistive sensing material due to its flexible and unsaturated coordination sphere around the Bismuth metal center which may act as a potential active site and accommodate gaseous probes, causing a direct perturbation of the charge mobility with the semiconductive network. There is also the presence of free, uncoordinated hydroxy groups in both Bi(HHTP)-α and Bi(HHTP)-β that can promote H-bonding interactions in the vicinity of the Bi atom. To characterize the ability of Bi(HHTP) to sense small reactive gases through electronic doping interactions, Applicants examined the chemiresistive responses of Bi(HHTP) toward both oxidizing (NO) and reducing (NH₃) gaseous analytes. To further probe Bi(HHTP)s capacity to detect analytes through a combination of electronic doping and H-bonding interactions, Applicants also examined the response of Bi(HHTP) with a range of H-bond donors (MeOH, EtOH, iPrOH) and H-bond acceptors (acetone).

To carry out the sensing procedure, Applicants dropcasted 10 μL of a Bi(HHTP) suspension (1-2 mg/mL in H₂O) onto 5 devices containing interdigitated 10 μm gap gold-electrodes, which generated devices with resistances in ˜30 MΩ range. The devices were then placed into an edge connector, wired to a breadboard and potentiostat (PalmSens) that applied 1.0 V voltage at room temperature. The devices were then enclosed in a Teflon chamber with gas inlet/outlet ports connected to Smart-Trak mass flow controllers delivering target concentrations of gases from pre-mixed tanks purchased from AirGas (tanks of 10,000 ppm of NH₃ in N₂, and 10,000 ppm of NO in N₂). The concentrations of the gaseous analytes were modified by adjusting flow rates (N₂ as the balance/purging gas). Generally, 5 devices at a time were exposed to one-minute saturations at different concentrations (5-1000 ppm) of the chosen analyte at a N₂ flow rate of 0.5 L/min and then purged with dry N₂ for 5 minutes to examine Bi(HHTP) recovery.

For VOC sensing, a Kintek FlexStream gas generator was used to produce vapors of the analyte (ethanol, methanol, acetone, or isopropanol), which was diluted in N₂ (4 L/min) to the desired concentration. Each organic vapor was calibrated before use in the generator by heating the internal permeation glass chamber/tube through loading a vial of the desired VOC and setting the span flow rate at for N₂ at 4 L/min. Notably, Applicants observed that altering the flow rates between analytes effects the response of the material, where higher flow rates are used to deliver lower concentrations, thus Applicants chose to keep the flow rate constant and vary the temperature to acquire concentration-dependent experiments.

### Example 1.10. Chemiresistive Sensing Response

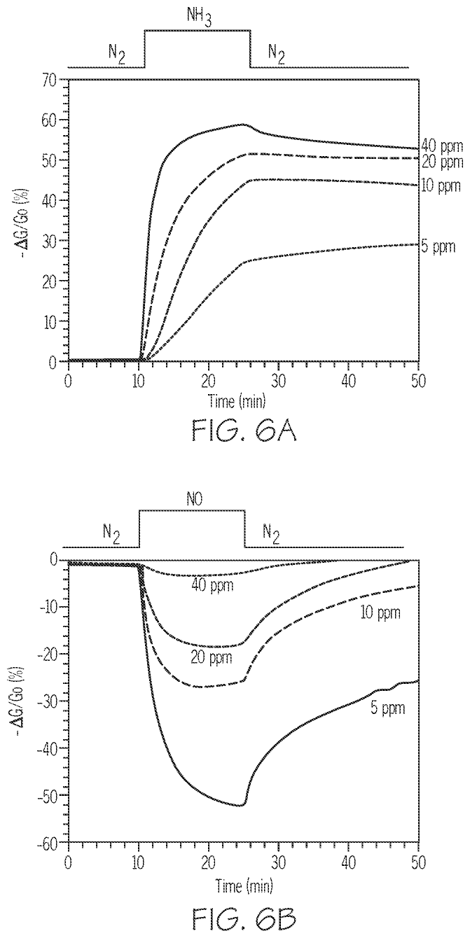

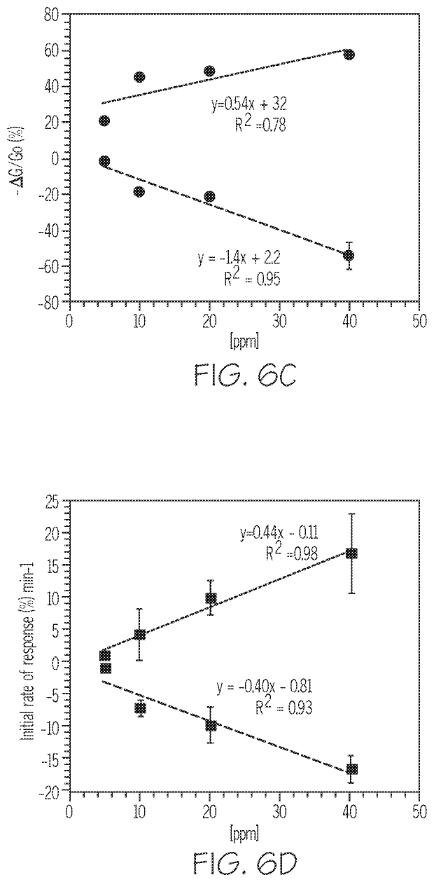

Although many examples of MOF-based sensors exist, to the best of Applicants' knowledge this report constitutes the first example of bismuth-based chemiresistive sensing. The favorable semiconductive nature of Bi(HHTP) facilitated the integration of Bi(HHTP) into devices through dropcasting and examine the chemiresistive response of Bi(HHTP) to the four VOCs (acetone, ethanol, methanol, and isopropanol) and 40, 20, 10 and 5 ppm of NO and NH₃. Bi(HHTP) exhibited a decrease in conductivity to the reducing gas (NH₃) and an increase in conductivity to the oxidizing gas NO (FIGS. 6A-6D).

Upon exposure to 40 ppm of NO, Bi(HHTP) showed a normalized response (−ΔG/Gₒ) of −54.8±6% after 15 minutes of exposure with reversibility. The normalized response (−ΔG/Gₒ) to NH₃ of 58.4±2% after the first 15 minutes of exposure. The observed chemiresistive responses to both oxidizing and reducing gases are thus consistent with the response of a p-type semiconductor. Applicants also examined the response of Bi(HHTP) to NO and NH₃ in the presence of humidity (5000 ppm of H₂O). Applicants observed a significant decrease in response in humidity (from −34.4±3.2% to −19.9%±0.76%−ΔG/Gₒ) when sensing NO and a considerable increase in response for NH₃ (from 39.6%±7.0% to −81.8%±7.3%−ΔG/Gₒ) in the same concentration of H₂O. Without being bound by theory, these results may point to the importance of the presence of hydrogen bonding in the sensing mechanism of NH₃.

Bi(HHTP) devices exhibit unique chemiresistive responses toward VOCs that change in direction of normalized conductance depending on the analyte. Both MeOH and acetone displayed an increase in normalized conductance (−ΔG/Gₒ) upon exposure, while ethanol and isopropanol demonstrated a decrease in normalized conductance (−ΔG/Gₒ) upon exposure to specific concentrations of analyte. All exposures to the VOCs were observed to be reversible. To better understand the responses and H-bonding interactions of Bi(HHTP) with the four VOCs, Applicants compared the pKas, dipoles moment and dielectric constants of each compound. The pKas of the VOCs increase from methanol to acetone. While ethanol and isopropanol have similar dipole moments, (1.66D), methanol and acetone have higher dipole moments. Other considerations include dielectric constants (F) which decrease from methanol, ethanol, isopropanol, and acetone, respectively. The combination of these electronic and structural properties may explain the observations noted during sensing of VOCs.

Furthermore, the presence of water molecules in the pores of Bi(HHTP)-β, as demonstrated by MicroED, may compete as host sites for hydrogen bonding with VOCs. Thus, the sensing responses to VOCs may have contributions from two competing mechanisms, one that involves hydrogen bonding on the surface of Bi(HHTP).

### Example 1.11. Limits of Detection

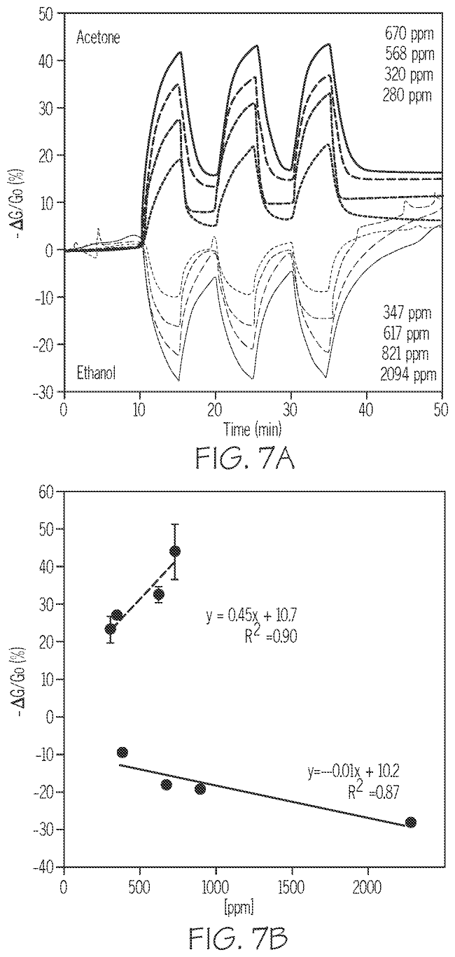

To examine the limits of detection (LODs), Applicants focused on two representative biomarkers that are known to be common breath metabolites, acetone and ethanol (vide infra). Applicants varied the concentration of these VOCs by increasing the temperature of the chamber housing the inner glass vial from 25° C.-40° C. and recorded three sequential exposures (FIGS. 7A-7B). Bi(HHTP) had an average response of 43.8±7% to 670 ppm of acetone after averaging across three devices exposed for 5 minutes and recovered in N₂ for 5 minutes, sequentially. To 2094 ppm of ethanol, Bi(HHTP) has an average response of −28.5±2%.

To determine the limits of detection (LODs) in response to NO and NH₃, Applicants calculated the change in response of Bi(HHTP) upon 15 minutes of exposure towards NO at different concentrations (5-40 ppm). The theoretical LODs are calculated based on the response after 15 minutes of exposure to either NO or NH₃ (5 to 40 ppm) were 0.15 ppm and 0.29 ppm, respectively. These LOD values are comparable to M₃(HXTP)₂-based systems, but do not exceed previously reported MPc-based 2D framework sensitivity to NO. Here however, Bi(HHTP) displays a unique reversibility to NO that is not observed in either of these previous systems. These reversible sorption characteristics are particularly advantageous for nanomaterial-based sensors that can be fabricated to withstand repeated exposures to NO for further enhancing long term. For VOCs, the LOD value for acetone is 41.2 ppm, 278 ppm for methanol, 50.2 ppm for isopropanol and 185 ppm for ethanol, which are similar to other reported chemiresistive values for alcohol sensors fabricated from metal oxides or reduced graphene oxides.

### Example 1.12. Sensing Mechanism Studies with NO Using XPS, ATR-IR, EPR and MicroED

To investigate the sensing mechanism, Attenuated Total Reflectance-infrared spectroscopy, (ATR-IR), X-ray Photoelectron spectroscopy (XPS) and Electron Paramagnetic Resonance (EPR) spectroscopy were used. ATR-IR can help visualize changes and shifts in modes of characteristic vibrational frequencies that indicate of variations in the coordination sphere and strength of specific bonds. XPS was used to confirm the elemental composition of Bi(HHTP), as well as identify chemical shifts typically associated with changes in population of electronic states. EPR allows the observation of analyte binding on the location and population of unpaired spins and/or changes in the oxidation state of metal and ligand constituents within the bulk material. As microED analysis was used for structure elucidation, Applicants speculated that this technique may be capitalized to view new or variations in electron density within the coordination network after gas exposure.

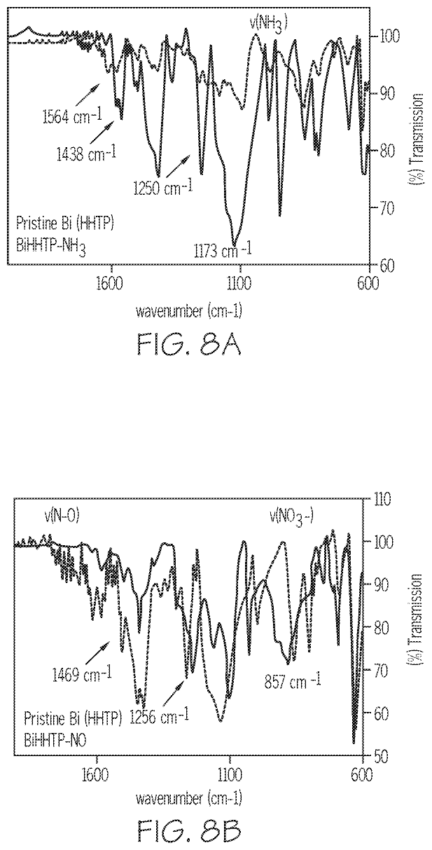

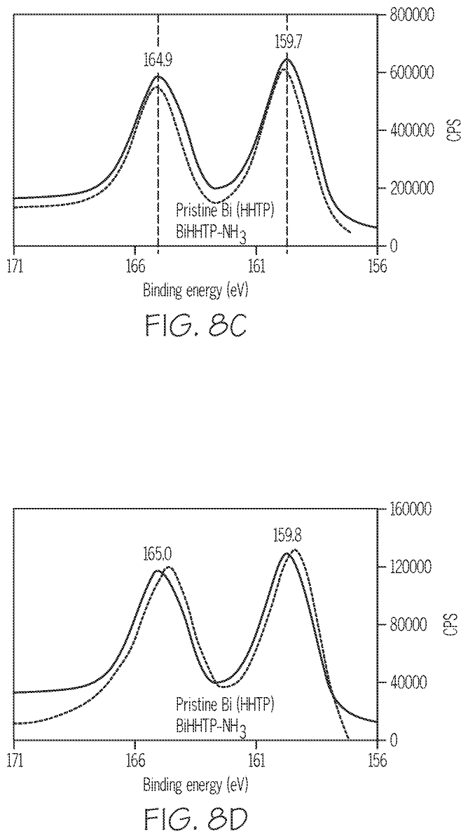

For ATR-IR analysis, pristine Bi(HHTP) was prepared by placing 20 mg of powder into a 2 mL vial and degassing with N₂ (1 L/min) for 2 hours. Analyte-exposed materials were prepared by purging the same batch of Bi(HHTP) with NO (1% of 10000 ppm tank) for 1 hour. Applicants used high concentrations of the analyte to ensure saturation of each sample to observe small detectable differences using XPS or IR. The samples were then sealed under an atmosphere of either N₂ or 1% analyte (balanced with N₂) and left overnight before measurement. ATR-IR revealed that after exposure to high concentrations of NO, the shifting of the peaks assigned to the Bismuth-catechol vibrational frequencies at 1256 cm⁻¹ and 1426 cm⁻¹ (FIG. 8B). This result suggests that the analyte-material interaction is occurring near or at the bismuth metal center, affecting the vibrational frequencies of the semiquinone ligand. IR analysis also revealed new bands occurring near 1500 cm⁻¹ (FIG. 8B), which indicate the creation of some ionic nitrate (NO₃⁻) species. This nitrite species could be generated from NO reaction with surface-bound O₂ on the coordination network. IR analysis of NH₃-exposed Bi(HHTP) revealed the presence of new bands present at approximately 1564 cm, 1438 cm⁻¹ and 1173 cm⁻¹ (FIG. 8A), which may arise from the contribution of Lewis acid site (LAS) interaction at the Bi center, given Bi(III) complexes are known to have good affinity for nitrogen donor ligands and Bi(III) borderline “hard” Lewis acidity. The symmetric stretching mode of NH₃ gas was also observed at 950 cm⁻¹. There may also be an indication of hydrogen bonding or interaction using Brønsted acid sites (BAS) of ammonia with uncoordinated hydroxy groups suggestive of the bands arising near 1250-1050 cm⁻¹.

XPS comparative analysis (carried out at 10⁻⁹ Torr) was used to analyze the composition of the Bi(HHTP) in its pristine state and after exposure to NO and NH₃. First, a pristine sample of Bi(HHTP) was purged for one hour with N₂, while another batch was saturated with NO or NH₃ (1%, 10,000 ppm) for one hour and sealed (left for over 24 hours as samples were shipped out for analysis. High-resolution deconvoluted spectra of the carbon 1 region after NO dosing revealed an increase in the peak assigned to the C—O—Bi binding energy and decrease in the peak area corresponding to the C═O—Bi binding energy, which supports the hypothesis that the interaction is occurring at the Bi node is causing a shift in the chemical environment near the semiquinone region. Although not further oxidized, the deconvoluted region of Bi4f_{7/2} and the Bi4f_{5/2} in the NO-doped Bi(HHTP) displayed a slight shift toward higher binding energies (FIG. 8F). This shift may be attributed to electron density transferring from the Bi center to the ligand and/or guest analyte, causing higher binding energies of more tightly bound emitted electrons. It is important to note that due to the high vacuum conditions under which XPS data was obtained and the reversible response of Bi(HHTP) to NO understood from previous sensing experiments, the aspect of XPS analysis may have promoted desorption of NO from the material, leading to an altered electronic state during sensing than the one observed during XPS. For NH₃ exposure, the C is spectrum displayed a slight increase in the area corresponding to the C═O—Bi bond, and in the region corresponding to the C—O—Bi bond. The high-resolution spectrum of the Bi 4f orbitals also indicated a small shift of the Bi4f7/2 and the Bi4f5/2 peaks, indicating an increase in electron density near the Bi metal node.

To supplement the spectroscopic investigations of the sensing mechanism, Applicants used microED to analyze structural or electronic density changes in Bi(HHTP) induced by exposure to NO and NH₃ (exposed for one hour at high concentration, 10,000 ppm or 1% of gas tank). Applicants observed that, after analyte exposure, Bi(HHTP)-NO displayed distinctly different structure and unit cell parameters, while Bi(HHTP)-NH₃ displayed a similar structure to that of Bi(HHTP)-β. Furthermore, the unit cell parameters varied considerably from that of both polymorphic structures.

Without being bound by theory, Applicants hypothesize that this change may have been induced by either occupation of the pores within the coordination network or through structural changes induced by analyte interaction with the host sites within the network. To confirm these structural changes induced by analyte exposure, Applicants utilized pXRD analysis on samples before and after one hour exposure to 10,000 ppm of NH₃ and NO. After NH₃ exposure, Bi(HHTP) exhibited a significant shift in the peak corresponding the (321̅) plane. This plane runs parallel to the π-π stacking layers, which indicates that ammonia exposure is resulting in the increasing of distances within these planes. This could result from ammonia occupying the available void volumes within Bi(HHTP) and on the edge sites of the structure, causing the expansion and increase in spacing of these layers. After recovery in N₂ for two hours, this shift did not return to its original peak position, consistent with Applicants' observations in sensing that NH₃ binds irreversibly to Bi(HHTP). For NO exposure, Applicants observed a slight shift in the (002), (200), (202̅) and (321̅) planes. This peak shift partially recovers after N₂ exposure for two hours (Applicants believe observing partial recovery is due to exposure to extremely high concentrations of NO), which is consistent with Applicants' observation in sensing that NO binding is weak and reversible. These slight deviations in peak position could also indicate NO occupying the available volume within the pores of Bi(HHTP), which is feasible considering the bond length of NO (1.15 Å), causing increases in distances between Bragg planes.

Electron paramagnetic resonance spectroscopy of the pristine Bi(HHTP) material displayed a broad absorbance band with low intensity centered at g=2.0, which may indicate an unpaired electron residing primarily in a ligand-centered orbital or possibly oxygen located on defect sites within the material. Only a slight increase in the intensity of the resonant absorbance was observed with exposure to NO, which is consistent with Applicants' observations from sensing that the interaction of the material with NO is weak and reversible. This slight increase was also observed in marginally higher intensity for the NH₃-exposed Bi(HHTP). This result suggests that NH₃ induced a slight change in the coordination sphere around the EPR active centers, possibly on the ligand.

For the VOC analytes, Applicants speculate that exposure to both MeOH and acetone result in the depletion of charge carriers (holes) through either electron transfer, hydrogen bonding or proton-coupled electron transfer interactions. The opposite response is observed for EtOH and IspOH, where the creation of charge carriers (holes) is observed upon exposure to these two VOCs. These differences in sensing response may result from steric differences within the VOCs, where MeOH and acetone gain easier access to host sites that are inaccessible to EtOH and IspOH due to steric affects and van der Waals volume. After MicroED analysis indicated the possible presence of water within the pores and coordinated to one of the Bi atoms within Bi(HHTP)-β, another possibility explaining the sensing observation is ethanol and isopropanol hydrogen bonding to water molecules and displacing their positions within the pores, triggering a structural change that promotes the mobility of charge carries within the network.

To summarize, XPS and IR analysis indicate that exposure of Bi(HHTP) to NO and NH₃ designate a significant variation in the electronic state of the ligand and bismuth node. Although the Bi center was not further oxidized, a shift in the Bi4f_{7/2} and the Bi4f₅/2 peaks indicated a shift in electron density away from the Bi center after exposure to NO, while NH₃ exposure suggested a slight increase in density at the Bi site. Due to the strong and irreversible binding of the analyte NH₃ within the network, Applicants were able to observe the presence of a N is peak in the XPS spectrum. IR analysis revealed the presence of new bands that correspond to Lewis acid interactions where binding is strong and irreversible, consistent with what Applicants observe in sensing. Taken together, both XPS and ATR-IR indicate analyte interaction occurring at or near the coordination sphere of Bi(HHTP) for the NO and NH₃ analytes.

To Applicants' knowledge, this Example constitutes the first demonstration of a bismuth-based coordination polymer towards chemiresistive sensing. To the best of Applicants' knowledge, Bi(HHTP) is among the first HHTP-based network structures solved using electron diffraction techniques. While microED was initially popularized for biological materials, this technique has proven invaluable for the structural elucidation of nanometer-sized crystalline materials often exhibited in MOFs or CNs. Bi(HHTP) is connected using polyaromatic 2,3,6,7,10,11-hexahydroxytriphenylene (HHTP) ligands interconnected with bismuth metal nodes and exhibits an unprecedented network topology with intricately connected layers, along with good electrical conductivity (5.3×10⁻³ S·cm⁻¹) when compared to other HHTP based 2D MOFs. Bi(HHTP) can also be synthesized at room temperature in an environmentally friendly aqueous environment using relatively inexpensive starting materials. Compared to other reported Bismuth-based MOFs that are commonly linked using polyaromatic carboxylate linkers and secondary-building units that exhibit larger pore apertures, Bi(HHTP) adopts a similar herring bone packing (similar to HHTP packing) with slit-like pores.

Applicants demonstrate the utility of this material toward chemical sensing of NO and NH₃ with limits of detection of 0.15 and 0.29 ppm, low driving voltages (0.1-1.0 V), and operation at room temperature. The LOD values for NO and NH₃ are similar to those reported using first-row transition metal HHTP-based 2D MOF sensors, and rival that of 2D MOFs made using layer-by-layer liquid-phase epitaxial techniques, but are not as sensitive as MPc-based 2D MOFs in terms of sensing NO. What is particularly noteworthy is Bi(HHTP)s unique and promising selective and reversible response towards NO, seldom seen in chemiresistive sensing using conductive coordination networks. Limitations of Bi(HHTP) in the context of chemiresistive sensing may be centered on the limited control over the spatial orientation on the surfaces of devices and the thickness of the film. Applicants also demonstrate the utility Bi(HHTP) towards sensing four structurally analogous VOCs (acetone, methanol, ethanol and isopropanol) to exhibit unique and reversible responses.

This Example demonstrates the development of a new class of semiconductive crystalline materials using nodes with the ability to accommodate high coordination numbers, high electron density and adaptable coordination environments. This flexible coordination sphere can permit the examination of structure-property relationships of Bi using other symmetrical polyaromatic linkers with different heteroatoms. Applicants' work demonstrates that harnessing electronic doping combined with the possibility of H-bonding interactions can lead to unique responses to structurally analogous analytes with similar functional groups (e.g. alcohols). Furthermore, advancing the development of these materials can enable a new class of sensors with ambient operating temperatures, low driving voltages in devices, and enhanced selectivity towards specific analytes for optimized performance.

### Example 2. Conductive Textile-Integrated Coordination Networks Based on Bismuth for Simultaneous Protection and Detection of Ionizing Radiation

The development of new multifunctional materials capable of simultaneously detecting and attenuating ionizing radiation will enable space exploration and ensure safe conditions for an array of work environments. Although forms of ionizing radiation, such as X-rays and UV rays, are valuable in today's health and research, these energy sources are also hazardous to human health, posing as health threats when present in high volumes or high dosages.

Fields in which humans require close contact with harmful forms of radiation dosimeters and protective equipment have been valuable for mitigating negative effects of ionizing radiation in medical imaging, nuclear energy development, space exploration, as well as areas of radiological contamination. However, despite the progress in the development of dosimeters and personal protective equipment, multifunctional systems that have the simultaneous characteristics of detection and attenuation, coupled with the beneficial characteristics of durability, flexibility, and wearability possessed by garments are currently unavailable.

To be effective in shielding from ionizing radiation, radiation attenuating materials densely pack high atomic numbered elements (Z=56 or higher) which attenuate radiation through either elastic (Rayleigh) or inelastic (Compton) scattering interactions or, more advantageously, absorb the incident ionizing radiation via the photoelectric effect. Materials, such as lead, have been well established to protect against serious biological damage from ionizing radiation, due to its high atomic number and its densely packed crystal structure.

Conventional shielding methods against ionizing radiation are limited by three (3) drawbacks. First, commercially available radiation shielding personal protective equipment (PPE) primarily consists of lead plates placed within fabric sleeves on aprons, gloves, and other garments. To ensure sufficient shielding capacity for these materials, the lead plating is thick, leading to heavy PPE which can limit user dexterity as well as discomfort the user over extended periods of time.

Second, the radiation shielding elements are rigid and inflexible, thereby leading to achieve structural defects upon bending, which in turn reduces the radiation shielding element's attenuative properties. Third, nonlead plated materials rely upon doping fabrics and surfaces with high atomic number elemental nanoparticles, which presents manufacturing challenges, including the even dispersal of densely packed high atomic elements and resistance to leaching upon mechanical or chemical strain.

The current state of the art for solid state, resistive radiation detection primarily consists of semiconductive single crystal and metal oxide materials. The features which permit the dosimetric detection of radiation in these materials include the presence of a high atomic numbered element within the crystal network to promote durability against ionizing radiation, a moderately large band gap (1.5 eV) to avoid leakage current and loss of signal, and a dense, pure crystal structure to promote signal transport through a material. Additionally, the ability of this same material to act as a functional dosimeter that can warn wearers when in proximity of harmful doses of ionizing radiation can ensure the safety of the wearer when encountering areas of unknown radiological safety through characteristic radiation responses.

The shortcomings which prevent these radiation-detecting single crystals and metal oxide materials from being used as multifunctional, wearable dosimeters include 3 major drawbacks. First, to modulate band gap, attenuation, and other physical properties, radiation-detecting single crystals and metal oxides often utilize toxic, heavy elements (e.g., Cd, Pb, Sn, and/or Tl) within their frameworks, which is not feasible for use in PPE. To ensure charge carrier mobility within their full domain, single crystal detectors require the growth of large, pure crystals which can be challenging and limit the scalability of devices based on these detectors. Additionally, these materials are often placed within devices as thin films which lack mechanical durability, restricting movement of the user. Accordingly, the ability to generate a material that can directly integrate with a shielding protective suit, or a uniform fabric will greatly improve the wearers' capacity to safely operate near sources of ionizing radiation.

Current advancements in radiation attenuating thrusts include the integration of high atomic number nanoparticles including bismuth and other non-toxic nanoparticles into textiles and other polymeric substrates to generate lightweight, wearable radiation shielding PPE.

While textile integration of these nanoparticle-based materials has offered the properties of flexibility and durability while being lightweight, challenges with insufficient dispersal of heavy atoms for radiation shielding, limited stability at the point of use, and a lack of further functionality in response to external stimuli have overshadowed progresses made in radiation attenuative studies. Furthermore, to enhance the safety of radiation PPE, it is desirable to also develop these attenuating materials with the capacity to simultaneously attenuate and detect radiation in the environment.

This Example represents a novel design for a dual functioning attenuator and dosimeter textile which, to the knowledge of Applicants, represents the first textile integrated radiation dosimeter. Given the shortcomings of existing materials on the attenuation and detection of radiation, and lack of available materials for the simultaneous textile integrated function, Applicants reasoned that metal-organic frameworks (MOFs) with properties of flexible coordination spheres within the metal node and organic linking molecule, good, p-type conductivity, and high atomic number metal centers deposited onto fabric substrates would be suitable candidates to bridge this gap in capabilities.

### Example 2.1. Experimental Design for Bi(HHTP)-Based MOFs

In recent years, MOFs and coordination networks have proved promising in sensing fields and PPE development through gains in chemiresistive sensing, electrochemical detection, and electromagnetic radiation.

Gains in sensing are owed to strategic molecular design choices in the metal centers, organic linkers, and applicability in an array of device designs, including growth on textiles. Electronic textiles (e-textiles) containing conductive MOFs specifically have shown promise for PPE development due to their sustained, functional electronic properties while maintaining structural durability within a wearable garment. Because MOFs and coordination networks have metal nodes structurally built in, they ensure consistent dispersal of metal atoms throughout the bulk of the material.

The compounded porosity of MOFs within a porous textile system such as cotton or polyester ensures a lightweight garment, and the chemisorptive relationship between conductive MOFs and textile domains establishes a flexible and durable stimuli responsive surface.

MOF-based, resistive, radiation detection utilizes p-type semiconductive framework materials to detect radiation due to a conductive bridging ligand and a metal center. Per the protocol outlined in Example 1, a coordination network with good conductivity named bismuth 2,3,6,7,10,11-hexahydroxytriphenylene [Bi(HHTP)] was synthesized and characterized for its chemiresistive sensing capabilities.

As described in Example 1, Bi(HHTP) is a semiconductor, demonstrating a band gap of 1.61 eV with chemiresistive sensing capabilities. See Example 1 of the Application. Applicants chose Bi(HHTP) as a coordination network for a dual functioning radiation attenuator and detector because of the combined individual components as well as three (3) key molecular design properties: (1) Bi(HHTP) possess good, p-type conductivity due to favorable energetic and spatial overlap between the O2p and Bi6p orbitals; (2) Bi(HHTP) maintains a denser packing structure (26.8 m² g⁻¹ surface area) relative to MOFs, which is advantageous for lightweight attenuation systems; and (3) Bi(HHTP) has several flexible coordination environments within its two polymorphs, which promote chemisorptive opportunities through H-bonding and bismuth centered interactions.

Accordingly, bismuth shows promise as a metal node for textile-based PPE against radiation due to being relatively non-toxic and radioactively stable compared to other high atomic number attenuating elements, possessing a flexible coordination sphere for both coordination networks and chemisorptive interactions, and having a high atomic number for radiation attenuative purposes. HHTP's precedence for e-textile MOF growth, redox enhanced flexible coordination spheres leading to good conductivity, and proclivity to p-type charge transport through multiple redox states demonstrate its promise as an organic linker for a radiation attenuating and detecting material.

### Example 2.2. Selection of Cotton and Scuba as Textile Domains

Applicants chose woven cotton and woven scuba (90:10 blend of polyester and elastane) as textile platforms for e-textile devices due to a myriad of strategic advantages present in both domains. First, Applicants chose cotton due to its favorable qualities, including its ubiquity in garments and fabric-based applications, its highly porous and durable morphology, and its advantageous chemical composition, containing several moieties capable of interacting chemisorptively with Bi(HHTP) such as three hydrogen bond donors (hanging hydroxyl group and the trans, equatorial diol) and a hydrogen bond accepting group (ether oxygen).

Applicants chose scuba because of key advantages, including its scalable thickness which allow for direct comparisons in surface deposition and linear attenuation of radiation, coming in 1.5 mm thick and 3.0 mm thick, its polymeric blend of polyester and elastane which allows for both rigidity during the templating process and flexibility in the fibrous domains during applications, and its diverse collection of functional groups capable of chemisorptively binding with Bi(HHTP) including amides (carbonyl and amine H-bonding), esters (H-bond accepting ether and carbonyl), and ethers (H-bond acceptors).

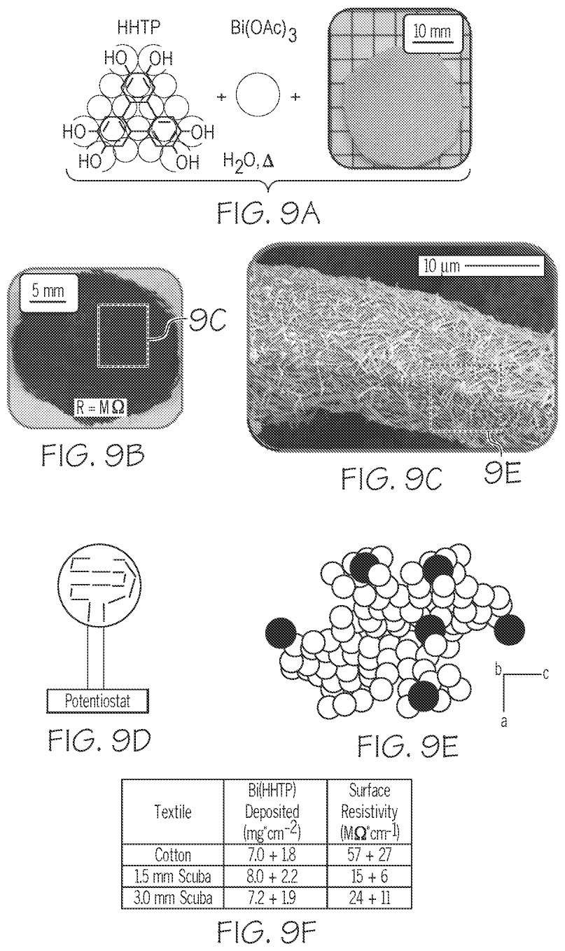

Applicants reasoned that, when deposited onto textiles, Bi(HHTP) would engender conductive properties to the textile when sufficient Bi(HHTP) grows on the surface of the material (FIG. 9B). Applicants grew Bi(HHTP) onto the cotton and scuba textile domains, generating conductive, green e-textiles, herein named Bi(HHTP) self-organized frameworks on textile [Bi(HHTP) SOFT] devices (FIGS. 9A-9F). Applicants utilized an in situ one-pot, hydrothermal, “Shake and bake” self-assembly synthetic method to chemisorptively deposit Bi(HHTP) onto both cotton and scuba swatches (FIG. 9A). The results demonstrated that Bi(HHTP) SOFT devices provide a valuable technological platform for achieving radiation attenuation with modular, lightweight, chemically precise, functional, and porous materials capable of being integrated into textiles (FIGS. 9A-9F).

### Example 2.3. Device Fabrication

To generate Bi(HHTP) SOFT devices, Applicants utilized an environmentally friendly, hydrothermal method with minimal synthetic intervention. Applicants began by cutting commercial cotton and scuba fabrics into 2 cm diameter circles (FIG. 9A) before adding them into a 20 mL scintillation vial with 10 mL of deionized water and a small stir-bar, ensuring the textile was fully submerged in solvent. Applicants then added equimolar quantities (0.45 mM) of bismuth acetate [Bi(OAc)₃] and HHTP to the vessel before the mixtures were subjected to sonication (30 min) until the mixture was a Kelly-green colored solution and stirred overnight at 85° C.

The resulting Bi(HHTP) SOFT devices cooled to room temperature before Applicants isolated and dried them under reduced pressure at 52° C. After drying for 48 h, Applicants rinsed the swatches with water (3×20 mL) and acetone (2×20 mL) before drying the devices under reduced pressure.

Applicants determined that the devices could be subjected to a second hydrothermal deposition to enhance the mass of Bi(HHTP) deposited and the homogeneity of Bi(HHTP) surface coverage. The resulting Bi(HHTP) SOFT devices comprised an average of 41% to 17% Bi(HHTP) by weight for cotton and both scuba domains, respectively. This corresponds to a range from 0.22 g/cm³ to 0.05 g/cm³ of Bi(HHTP) within the SOFT devices.

SOFT-supported Bi(HHTP) constituted 11% to 12% of the total mass of Bi(HHTP) prepared, considering both the SOFT-device and the precipitated Bi(HHTP) powder isolated from the reaction solution. Through direct solution-phase crystallization of Bi(HHTP), Applicants could generate a scalable surface area of conductivity, ranging from 6.3 cm² up to 16 cm² (measured diagonally across a 4×4 cm swatch) of megaohm scale resistive surfaces. This fabrication method yielded functional swatches with moderate sheet resistance (ranging from 20±6 MΩ for Bi(HHTP) SOFT cotton devices to 10±4 MΩ and 5±1 MΩ for 3.0- and 1.5-mm thick scuba, respectively) and high yields of functional devices.

For each device, Applicants performed a minimum of n=10 fabrication trials, yielding functional devices for every device for Bi(HHTP) on cotton (n=13), on 1.5 mm scuba (n=13) and on 3.0 mm scuba (n=14).

### Example 2.4. Material Characterization and Analysis

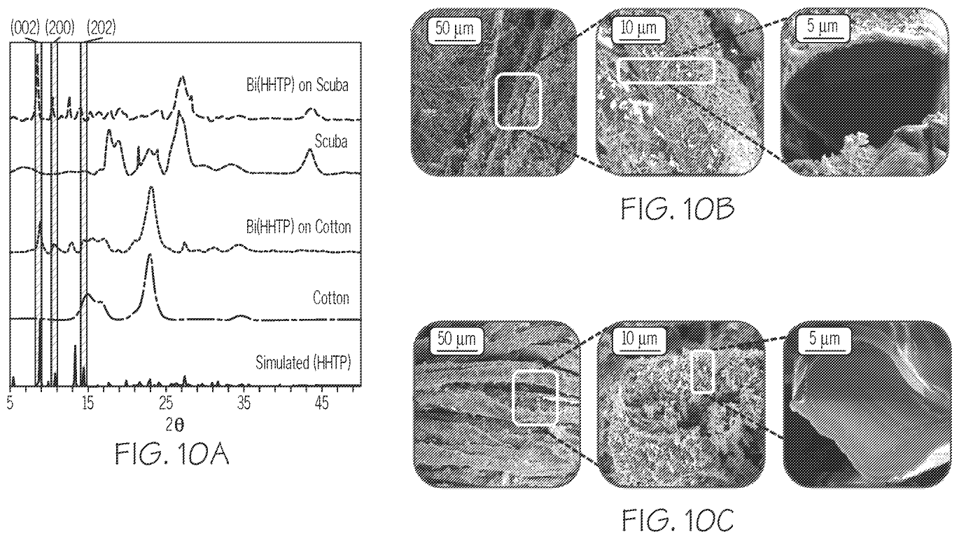

To assess the presence of crystalline Bi(HHTP) on Bi(HHTP) SOFT devices, Applicants took powder x-ray diffractometry (PXRD) to compare SOFT devices both Bi(HHTP) simated from a cif file and blank textiles. Bi(HHTP)'s structure was solved in the studies described in Example 1, which allowed Applicants to assess Bi(HHTP) templated onto cotton and scuba (FIG. 10A). The PXRD spectra shows the characteristic (002), (200), and (202) peaks at 20 values of 8.36°, 10.82°, and 14.46° respectively. In order, these peaks are correlated to interatomic distances of 10.6, 8.0, and 5.9 Å, according to Bragg's law. The (002) and (202) planes of Bi(HHTP) bisect one HHTP when viewed along the crystallographic c-axis, and the (200) plane intersected and ran parallel to the metal nodes. Some slight peak shifting is observed in characteristic Bi(HHTP) peaks due to the height of the Bi(HHTP) deposited onto the textiles when compared to the simulated Bi(HHTP) spectrum.

### Example 2.5. Morphological Analysis

Scanning electron microscopy (SEM) and focused ion beam (FIB) characterization showed uniform dispersal of Bi(HHTP) throughout the textile domains and confirmed Bi(HHTP)'s rod-like morphology previously reported in Example 1 (FIGS. 10B-10C). Energy dispersive spectroscopy (EDS) mapping showed even dispersal of bismuth, carbon, and oxygen while EDS spectra agreed with Bi(HHTP)'s relative elemental stoichiometric ratios. Focused Ion Beam (FIB) images (FIGS. 10B-10C) illustrated conformal coating of textile fibers by Bi(HHTP) nanorods, with favorable adhesion along the long edge of the rod. These FIB images showed that the Bi(HHTP) grows in a rod like morphology flat to the surface of the threads.

### Example 2.6. ATR-IR Characterization of Bi(HHTP) Growth on Textile Fibers

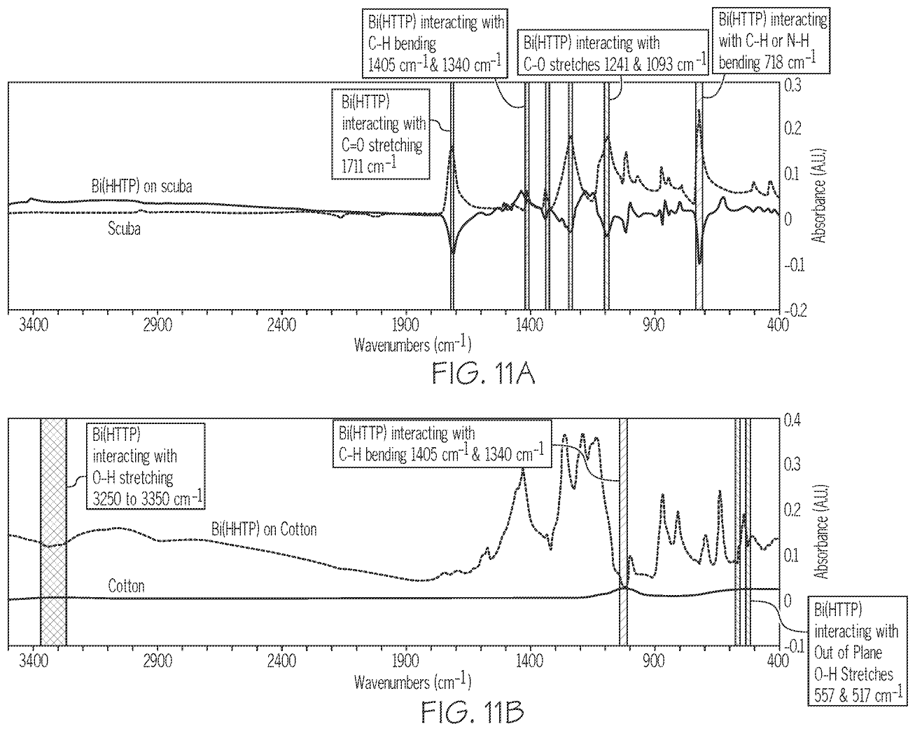

Applicants used attenuated total reflectance infrared spectroscopy (ATR-IR) to determine the nature of the surface level chemisorptive interactions between Bi(HHTP) and both cotton and scuba. Both cotton and scuba contained key functional groups with ATR-IR signals capable of interacting with Bi(HHTP) chemisorptively (amides, esters, alcohols, ketones, and ethers). Applicants probed the difference in the absorption spectra of Bi(HHTP) deposited on the textile substrate and the textile substrate itself, characterizing these interactions as disruptions in the textile's electronic structure leading to reversals in IR band-structures, seen in FIGS. 11A-11B. Bi(HHTP) on cotton showed a reversal of the O—H stretching band around 3300 cm⁻¹, which could be connected to Bi(HHTP) through either hydrogen bonding to cellulose or unsaturated bismuth nodes coordinating to these hydroxyls. Bi(HHTP) also showed a reversal of the cotton band structure around the 1050 to 983 cm⁻¹ range, and this reversal may be related to the C—O stretches in the ether moiety of cotton. This disruption may arise from hydrogen bonding interactions with the cotton molecular structure.

Bi(HHTP) on scuba showed a paired reversal of comparable peaks located at 1711 and 718 cm⁻¹. These peaks are related to both the carbonyl stretching mode and likely an N—H bending mode, respectively. Applicants hypothesize that this is related to Bi(HHTP) chemically bonding to the carbonyl moiety with either or both HHTP-based hydrogen bonding or through an unsaturated bismuth node and bonding to the N—H through hydrogen bonding. Bi(HHTP) SOFT on scuba showed a greater number of disruptions in the IR spectrum than the SOFT-cotton related spectrum. This observation suggests that Bi(HHTP) may have greater chemisorption to the surface of scuba than the surface of cotton.

### Example 2.7. Mechanical Stability/Bi(HHTP) Resiliency

The Bi(HHTP) SOFT devices maintained their physical textile form after deposition and subsequent washing and could bend and twist without losing a significant mass of deposited framework material or surface resistivity. Bi(HHTP) SOFT devices could be handled gently (with forceps, voltmeter probes on surface, pinching) without measurable loss of Bi(HHTP) mass. Bi(HHTP) SOFT cotton devices lost more mass during handling than scuba devices due to the growth of shards/chunks of Bi(HHTP) on the surface of cotton devices. Radiation sensing and attenuation studies did not impact the Bi(HHTP) mass loading, nor did it impact the surface crystallinity.

A surface cohesion peel test was performed on Bi(HHTP) SOFT devices using commercially available double-sided tape to compare the cohesive interactions between Bi(HHTP) layers and the surface of cotton and scuba. Ensuring maximal surface contact on both sides of samples, strips of tape were pressed on and peeled off, and the resulting differences in mass for the textile and tape, as well as the losses in surface resistivity, were recorded. The results suggested that Bi(HHTP) cohesive forces on cotton and scuba were greater than cohesive forces between Bi(HHTP) and tape. Only 3.0 mm scuba SOFT samples showed a significant loss of Bi(HHTP) after cohesion testing, losing 1.80±0.01% of its Bi(HHTP) mass. However, the cohesion test did disrupt the surface resistivity of SOFT samples significantly for cotton and 3.0 mm scuba with deductions in surface resistivities of 30±18 MΩ and 16±4 MΩ, respectively. Without being bound by theory, Applicant hypothesize that this relationship between surface cohesion and surface resistivity may emphasize the through-bond charge transport nature of Bi(HHTP) within Bi(HHTP) SOFT devices.

### Example 2.8. Radiation Attenuation

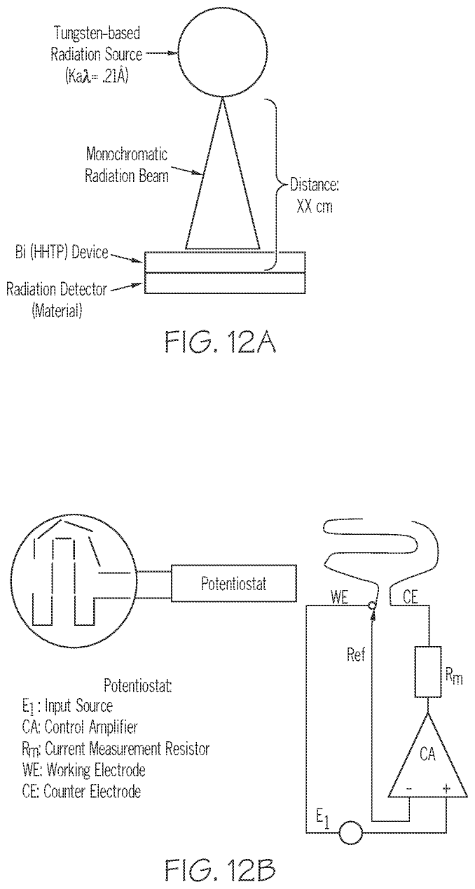

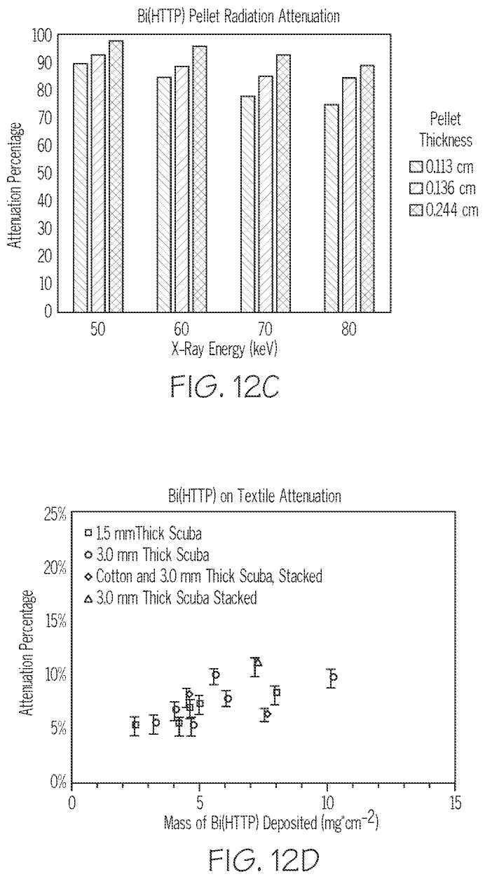

X-Ray attenuation was tested by using a Philips Digital Diagnostic Medical Grade X-Ray Unit (Andover, MA) equipped with a Tungsten X-Ray source. The detectors operate with 4 stacked solid-state, high purity silicon diode detectors with Cu and Al as filters and assembly is glued together with Ag-epoxy and bonded with gold wires. These RaySafeX2 detectors are calibrated to NIST standards and the system is enclosed in a tin cage that acts like a shield from scatter and a collimator. The results are summarized in FIGS. 12A-12D. Bi(HHTP) pellets and textiles templated with Bi(HHTP) were placed directly on top of the RaySafe X2 detector while a second RaySafe X2 detector remained uncovered to standardize the flux of radiation delivered by the X-Ray source. The percentage of attenuation for each Bi(HHTP) templated textile or combination of Bi(HHTP) templated textiles was calculated by comparing the RaySafe X2 detector with the Bi(HHTP) templated textile and the RaySafe X2 detector without any textile.

Applicants evaluated the attenuative capacity of the Bi(HHTP) pellets and SOFT devices by plugging in the initial intensity (I₀) and the attenuated intensity (I) into Equation 1 and comparing the resulting linear coefficient, μ in accordance with Equation 1.

I=Iₒe^{-μX} Equation 1

A derivation of Beer's Law (Equation 1) demonstrates the relationship between the Bi(HHTP) and the textiles in relation to X-Ray radiation where I represents the intensity of the attenuated beam of radiation, Iₒ represents the initial intensity of the unattenuated radiation beam photons passing through the material, and X represents the thickness of the attenuating material.

By assuming that all X-Ray shielding from samples was due to Bi(HHTP) particle size and the packing of bismuth atoms within Bi(HHTP) normal to the incident radiation, Applicants compared attenuation percentages

$$( \frac{I}{{I}_{0}} )$$

as the representation of a material's capacity to attenuate radiation in the solid state.

Applicants saw that X-Ray attenuation percentages (FIG. 12C) reached up to 90% across the range of radiation for Bi(HHTP) pellets and up to 17% for Bi(HHTP) SOFT against 60 kVp radiation. Applicants also observed a direct relationship between the thickness and mass loading of the Bi(HHTP) pellet or SOFT e-textile stacking and the radiation attenuation percentage.

This relationship was attributed to the increase in material thickness, χ, as well as the increase in bismuth atoms normal to the radiation source. Low radiation attenuation percentages for Bi(HHTP) SOFT devices were attributed to the low spatial distribution and stoichiometric quantity of bismuth within the Bi(HHTP) framework material. Previous radiation attenuation studies performed on nanoparticle doped textiles or polymers demonstrated greater attenuation percentages at comparable mass loading percentages.

### Example 2.9. Summary

In sum, Applicants successfully deposited Bi(HHTP) onto two woven textile domains, scuba and cotton, through a green, one pot, hydrothermal, self-assembly method yielding e-textile devices named Bi(HHTP) SOFT. Synthetic optimization processes have provided a method with reliable e-textile development and retrieval of non-deposited Bi(HHTP). Bi(HHTP) was characterized using PXRD, SEM, EDS, and FIB to demonstrate the crystalline growth of Bi(HHTP) nanorods on the surface of both cotton and scuba textile swatches.

Further characterization using ATR-IR and XPS illustrated the chemisorptive interactions between Bi(HHTP) and functional groups present on the textile surface. Additionally, Bi(HHTP) SOFT devices were proven to be durable against cohesion tests.

Both Bi(HHTP) bulk pellets and Bi(HHTP) SOFT devices were tested for their radiation attenuation and detection capabilities with favorable (≈90%) attenuation percentages against a diagnostic range of X-Ray radiation for bulk pellet materials and low attenuation percentages for Bi(HHTP) SOFT devices. These low e-textile attenuation values are accounted for by the porous nature of both the textile domain and the coordination network.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition comprising:
a metal-organic framework,
wherein the metal-organic framework comprises a plurality of metals and a plurality of ligands coordinated with the plurality of metals,
wherein the plurality of metals comprise bismuth,
wherein the plurality of ligands comprise a plurality of hydroxy moieties that are not part of carboxyl groups,
wherein at least some of the hydroxy moieties are coordinated with bismuth to form Bi—O bonds, and
wherein the metal-organic framework is in the form of a conductive and interconnected network.

2. The composition of claim 1, wherein the plurality of metals consist essentially of bismuth.

3. The composition of claim 1, wherein at least some of the hydroxy moieties form hydrogen bonds with water molecules.

4. The composition of claim 1, wherein the metal-organic framework comprises the following formula:
M_{y}(HXTP)_{z},
wherein M comprises bismuth,
wherein X is O or NH,
wherein y is 1 or 3,
wherein z is 1 or 2, and
wherein HXTP represents a triphenylene-based ligand selected from the group consisting of 2,3,5,6,10,11-hexahydroxytriphenylene (HHTP), 2,3,5,6,10,11 hexaiminotriphenylene (HITP), 2,3,5,6,10,11-hexathiotriphenylene (HTTP), and combinations thereof.

5. The composition of claim 4, wherein the metal-organic framework comprises Bi(HHTP)-α, wherein Bi(HHTP)-α comprises a 2,3-c nodal net stoichiometry.

6. The composition of claim 4, wherein the metal-organic framework comprises Bi(HHTP)-β, wherein Bi(HHTP)-β comprises a 3,4,4,5-c nodal net stoichiometry.

7. The composition of claim 1, wherein the metal-organic framework is a component of a sensor.

8. The composition of claim 1, wherein the metal-organic framework is a component of a dosimeter.

9. The composition of claim 1, wherein the metal-organic framework is associated with a textile, wherein the textile comprises a plurality of fibers and a plurality of pores, and wherein the metal-organic frameworks are associated with the fibers of the textile.

10. The composition of claim 1, wherein the metal-organic framework is a component of a personal protective equipment.

11. The composition of claim 1, wherein the metal-organic framework comprises a non-planar shape.

12. The composition of claim 11, wherein the non-planar shape comprises a three-dimensional shape, a polyhedral shape, a tetragonal pyramid shape, a quadrilateral shape, a layered shape, or combinations thereof.

13. The composition of claim 11, wherein the non-planar shape comprises a layered shape, wherein the layered shape is defined by a plurality of interconnected layers, and wherein the layers are interconnected through Bi—O bonds.

14. The composition of claim 1, wherein the metal-organic framework comprises a crystalline shape.

15. A method of detecting an analyte in a sample, said method comprising:
associating the sample with a composition,
wherein the composition is in the form of a sensor,
wherein the composition comprises a metal-organic framework,
wherein the metal-organic framework comprises a plurality of metals and
a plurality of ligands coordinated with the plurality of metals,
wherein the plurality of metals comprise bismuth,
wherein the plurality of ligands comprise a plurality of hydroxy moieties that are not part of carboxyl groups,
wherein at least some of the hydroxy moieties are coordinated with bismuth to form Bi—O bonds, and
wherein the metal-organic framework is in the form of a conductive and interconnected network; and
detecting the presence or absence of the analyte from the sample, wherein the detecting comprises:
detecting a change in a property of the sensor, and
correlating the change in the property to the presence or absence of the analyte.

16. The method of claim 15, wherein the change in the property of the sensor comprises a change in normalized conductance over time (ΔG/Go).

17. The method of claim 15, wherein the correlating comprises comparing the properties of the sensor to properties of the sensor in response to association with known analytes.

18. The method of claim 15, wherein the analyte is selected from the group consisting of gases, ketones, alcohols, aromatic compounds, volatile organic compounds, water, neurotransmitters, hormones, proteins, sugars, metal ions, ionizing radiation, NO, CO, H₂S, NH₃, H₂O, and combinations thereof.

19. The method of claim 15, wherein the analyte comprises one or more ionizing radiations selected from the group consisting of X-rays, UV-rays, or combinations thereof.

20. The method of claim 15, wherein the exposing results in the reversible association of the analyte in the sample with the composition.

21. The method of claim 20, wherein the association also results in the capture of the analyte by the sensor.

22. The method of claim 20, further comprising a step of releasing the analyte from the sensor.

23. The method of claim 20, wherein the metal-organic framework comprises the following formula:
M_{y}(HXTP)_{z},
wherein M comprises bismuth,
wherein X is O or NH,
wherein y is 1 or 3,
wherein z is 1 or 2, and
wherein HXTP represents a triphenylene-based ligand selected from the group consisting of 2,3,5,6,10,11-hexahydroxytriphenylene (HHTP), 2,3,5,6,10,11 hexaiminotriphenylene (HITP), 2,3,5,6,10,11-hexathiotriphenylene (HTTP), and combinations thereof.

24. The method of claim 23, wherein the metal-organic framework comprises Bi(HHTP)-α, wherein Bi(HHTP)-α comprises a 2,3-c nodal net stoichiometry.

25. The method of claim 23, wherein the metal-organic framework comprises Bi(HHTP)-β, wherein Bi(HHTP)-β comprises a 3,4,4,5-c nodal net stoichiometry.

26. A method of shielding an object from radiation, said method comprising:
associating the object with a composition, wherein the composition comprises a metal-organic framework,
wherein the metal-organic framework comprises a plurality of metals and a plurality of ligands coordinated with the plurality of metals,
wherein the plurality of metals comprise bismuth,
wherein the plurality of ligands comprise a plurality of hydroxy moieties that are not part of carboxyl groups,
wherein at least some of the hydroxy moieties are coordinated with bismuth to form Bi—O bonds, and
wherein the metal-organic framework is in the form of a conductive and interconnected network.

27. The method of claim 26, wherein the object is selected from the group consisting of an aircraft, a spacecraft, a space station, a human being, an animal, or combinations thereof.

28. The method of claim 26, wherein the associating occurs by a method selected from the group consisting of wrapping, adhering, surrounding, wearing, or combinations thereof.

29. The method of claim 26, wherein the object is a human being, wherein the composition is in the form of a personal protective equipment, and wherein the associating occurs by wearing the personal protective equipment.

30. The method of claim 26, wherein the radiation is selected from the group consisting of ionizing radiation, X-rays, UV rays, or combinations thereof.

31. The method of claim 26, wherein the metal-organic framework comprises the following formula:
M_{y}(HXTP)_{z},
wherein M comprises bismuth,
wherein X is O or NH,
wherein y is 1 or 3,
wherein z is 1 or 2, and
wherein HXTP represents a triphenylene-based ligand selected from the group consisting of 2,3,5,6,10,11-hexahydroxytriphenylene (HHTP), 2,3,5,6,10,11 hexaiminotriphenylene (HITP), 2,3,5,6,10,11-hexathiotriphenylene (HTTP), and combinations thereof.

32. The method of claim 31, wherein the metal-organic framework comprises Bi(HHTP)-α, wherein Bi(HHTP)-α comprises a 2,3-c nodal net stoichiometry.

33. The method of claim 31, wherein the metal-organic framework comprises Bi(HHTP)-β, wherein Bi(HHTP)-β comprises a 3,4,4,5-c nodal net stoichiometry.
